(19) United States
(12) Patent Application Publication
(10) Pub. No.: US 2008/0045824 A1
(43) Pub. Date: Feb. 21, 2008

(54) SILICONE COMPOSITION FOR BIOCOMPATIBLE MEMBRANE

(75) Inventors: Mark A. Tapsak, San Diego, CA (US); Paul Valint JR., Pittsford, NY (US)

Correspondence Address: KNOBBE MARTENS OLSON & BEAR LLP, 2040 MAIN STREET, FOURTEENTH FLOOR, IRVINE, CA, 92614, US

(73) Assignee: DexCom, Inc., San Diego, CA (US)

(21) Appl. No.: 11/763,215

(22) Filed: Jun. 14, 2007

Related U.S. Application Data
(62) Division of application No. 10/695,636, filed on Oct. 28, 2003, now abandoned.

(51) Int. Cl. A61B 5/1473

(52) U.S. Cl. 600/345

(57) ABSTRACT

The present invention relates generally to biosensor materials. More specifically, this invention relates to a novel polymeric material that can be useful as a biocompatible membrane for use in biosensor applications.

### RELATED APPLICATION

This application is a divisional of U.S. application Ser. No. 10/695,636, filed Oct. 28, 2003, which is incorporated herein by reference in its entirety.

### FIELD OF THE INVENTION

The present invention relates generally to biosensor materials. More specifically, this invention relates to a silicone polymeric material that can be useful as a biocompatible membrane for use in biosensor applications.

### BACKGROUND OF THE INVENTION

A biosensor is a device that uses biological recognition properties for the selective analysis of various analytes or biomolecules. Generally, the sensor produces a signal that is quantitatively related to the concentration of the analyte. In particular, a great deal of research has been directed toward the development of a glucose sensor that can function in vivo to monitor a patient's blood glucose level. One type of glucose sensor is the amperometric electrochemical glucose sensor. Typically, an electrochemical glucose sensor employs the use of a glucose oxidase enzyme to catalyze the reaction between glucose and oxygen and subsequently generate an electrical signal. The reaction catalyzed by glucose oxidase yields gluconic acid and hydrogen peroxide as shown in the reaction below (equation 1):

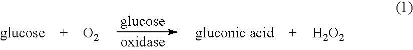

The hydrogen peroxide reacts electrochemically as shown below (equation 2):
H₂O₂→2H⁺+O₂+2e⁻ (2)

The current measured by the sensor is generated by the oxidation of the hydrogen peroxide at a platinum working electrode. According to equation 1, if there is excess oxygen for equation 1, then the hydrogen peroxide is stoichiometrically related to the amount of glucose that reacts with the enzyme. In this instance, the ultimate current is also proportional to the amount of glucose that reacts with the enzyme. However, if there is insufficient oxygen for all of the glucose to react with the enzyme, then the current will be proportional to the oxygen concentration, not the glucose concentration. For the glucose sensor to be useful, glucose is preferably the limiting reagent. The oxygen concentration is preferably in excess for all potential glucose concentrations. Unfortunately, this requirement cannot be easily achieved. For example, in the subcutaneous tissue the concentration of oxygen is much less that of glucose. As a consequence, oxygen can become a limiting reactant, giving rise to conditions associated with an oxygen deficit. Attempts have been made to circumvent this condition such that the sensor can continuously operate in an environment with an excess of oxygen.

Several attempts have been made to use membranes of various types to regulate the transport of oxygen and glucose to the sensing elements of glucose oxidase-based glucose sensors. For example, homogenous membranes having hydrophilic domains dispersed substantially throughout a hydrophobic matrix have been employed to facilitate glucose diffusion. For example, U.S. Pat. No. 5,322,063 to Allen et al. teaches that various compositions of hydrophilic polyurethanes can be used to control the ratios of the diffusion coefficients of oxygen to glucose in an implantable glucose sensor. In particular, various polyurethane compositions were synthesized that were capable of absorbing from 10 to 50% of their dry weight of water. The polyurethanes were rendered hydrophilic by incorporating polyethyleneoxide as their soft segment diols. One disadvantage of such materials is that the primary backbone structure of the polyurethane is sufficiently different such that more than one casting solvent may be required to fabricate the membranes. This reduces the ease with which the membranes may be manufactured and may further reduce the reproducibility of the membrane. Furthermore, neither the concentration of the polyethyleneoxide soft segments in the polymers nor the amount of water pickup of the polyurethanes disclosed by Allen directly correlate to the oxygen to glucose permeability ratios. Therefore, the oxygen to glucose permeability ratios cannot be predicted from the polymer composition. As a result, a large number of polymers must be synthesized and tested before a desired specific oxygen to glucose permeability ratio can be obtained.

U.S. Pat. Nos. 5,777,060 and 5,882,494 also disclose homogeneous membranes having hydrophilic domains dispersed throughout a hydrophobic matrix, which are fabricated to reduce the amount of glucose diffusion to the working electrode of a biosensor. For example, U.S. Pat. No. 5,882,494 discloses a membrane including the reaction products of a diisocyanate, a hydrophilic diol or diamine, and a silicone material. U.S. Pat. No. 5,777,060 discloses polymeric membranes that can be prepared from a diisocyanate, a hydrophilic polymer, a siloxane polymer having functional groups at the chain termini, and optionally a chain extender. Polymerization of these membranes typically requires heating of the reaction mixture for periods of time from one to four hours, depending on whether polymerization of the reactants is carried out in bulk or in a solvent system. Since the oxygen to glucose permeability ratios cannot be predicted from the polymer composition, a large number of polymers must be synthesized and coating or casting techniques optimized before desired specific oxygen-to-glucose permeability ratio could be obtained.

U.S. Pat. No. 6,200,772 discloses membranes with hydrophilic domains dispersed substantially throughout a hydrophobic matrix. The membranes limit the amount of glucose diffusing to a working electrode. In particular, the patent describes a sensor device that includes a membrane comprised of modified polyurethane that is substantially non-porous and incorporates a non-ionic surfactant as a modifier. The non-ionic surfactant can include a polyoxyalkylene chain, such as one derived from multiple units of polyoxyethylene groups. As described, the non-ionic surfactant may be incorporated into the polyurethane by admixture or through compounding to distribute it throughout the polyurethane.

PCT Application WO92/13271 describes an implantable fluid-measuring device for determining the presence and amounts of substances in a biological fluid. The device includes a membrane including a blend of two substantially similar polyurethane urea copolymers, one having a glucose permeability that is somewhat higher than the other.

### SUMMARY OF THE INVENTION

Biocompatible membranes and implantable devices incorporating such biocompatible membranes are provided.

In a first embodiment, a biocompatible membrane is provided, the biocompatible membrane comprising a silicone composition comprising a hydrophile covalently incorporated therein, wherein the biocompatible membrane controls the transport of an analyte through the membrane.

In an aspect of the first embodiment, the silicone composition comprises a hydrophile grafted therein.

In an aspect of the first embodiment, the biocompatible membrane comprises two or more domains.

In an aspect of the first embodiment, the biocompatible membrane comprises a cell disruptive domain, wherein the cell disruptive domain supports tissue ingrowth and interferes with barrier-cell layer formation.

In an aspect of the first embodiment, the cell disruptive domain comprises the silicone composition.

In an aspect of the first embodiment, the silicone composition comprises from about 1 to about 20 wt. % of the hydrophile.

In an aspect of the first embodiment, the biocompatible membrane comprises a cell impermeable domain, wherein the cell impermeable domain is resistant to cellular attachment and is impermeable to cells and cell processes.

In an aspect of the first embodiment, the cell impermeable domain comprises the silicone composition.

In an aspect of the first embodiment, the silicone composition comprises from about 1 to about 20 wt. % of the hydrophile.

In an aspect of the first embodiment, the biocompatible membrane comprises a resistance domain, wherein the resistance domain controls a flux of oxygen and glucose through the membrane.

In an aspect of the first embodiment, the resistance domain comprises the silicone composition.

In an aspect of the first embodiment, the silicone composition comprises from about 1 to about 20 wt. % of the hydrophile.

In an aspect of the first embodiment, the biocompatible membrane comprises an enzyme domain, wherein the enzyme domain comprises an immobilized enzyme.

In an aspect of the first embodiment, the immobilized enzyme comprises glucose oxidase.

In an aspect of the first embodiment, the enzyme domain comprises the silicone composition.

In an aspect of the first embodiment, the silicone composition comprises from about 1 to about 50 wt. % of the hydrophile.

In an aspect of the first embodiment, the biocompatible membrane comprises an interference domain, wherein the interference domain substantially prevents the penetration of one or more interferents into an electrolyte phase adjacent to an electrochemically reactive surface.

In an aspect of the first embodiment, the interference domain comprises an ionic component.

In an aspect of the first embodiment, the interference domain comprises the silicone composition.

In an aspect of the first embodiment, silicone composition comprises from about 1 to about 10 wt. % of the hydrophile.

In an aspect of the first embodiment, the biocompatible membrane comprises an electrolyte domain, wherein the electrolyte domain comprises a semipermeable coating that maintains hydrophilicity at an electrochemically reactive surface.

In an aspect of the first embodiment, the electrolyte domain comprises the silicone composition.

In an aspect of the first embodiment, silicone composition comprises from about 1 to about 50 wt. % of the hydrophile.

An implantable biosensor is provided comprising the bicompatible membrane of the first embodiment.

An implantable drug delivery device is provided comprising the bicompatible membrane of the first embodiment.

An implantable cell implantation device is provided comprising the bicompatible membrane of the first embodiment.

In a second embodiment, a polymeric material is provided, wherein the polymeric material comprises a repeating unit derived from a cyclosiloxane monomer substituted with a hydrophile, a repeating unit derived from an unsubstituted cyclosiloxane monomer, and a terminating unit derived from a polysiloxane monomer terminated with a telechelic group.

In an aspect of the second embodiment, the hydrophile comprises diethyleneglycol.

In an aspect of the second embodiment, the hydrophile comprises triethyleneglycol.

In an aspect of the second embodiment, the hydrophile comprises tetraethyleneglycol.

In an aspect of the second embodiment, the hydrophile comprises polyethyleneglycol.

In an aspect of the second embodiment, the polyethyleneglycol comprises from about 1 to about 30 repeating units.

In an aspect of the second embodiment, the unsubstituted cyclosiloxane monomer comprises octamethylcyclotetrasiloxane.

In an aspect of the second embodiment, the unsubstituted cyclosiloxane monomer comprises hexamethlcyclotrisiloxane.

In an aspect of the second embodiment, the unsubstituted cyclosiloxane monomer comprises octamethlcyclotrisiloxane.

In an aspect of the second embodiment, the polysiloxane monomer terminated with a telechelic group comprises a vinyldimethylsilyl-terminated polysiloxane.

In an aspect of the second embodiment, the polysiloxane monomer terminated with a telechelic group comprises a polydimethylsiloxane monomer terminated with a telechelic group.

In an aspect of the second embodiment, the polysiloxane monomer terminated with a telechelic group comprises divinyltetramethyl disiloxane.

In an aspect of the second embodiment, the divinyltetramethyl disiloxane comprises from about 1 to about 100 dimethylsiloxane units.

In an aspect of the second embodiment, the polymeric material comprises about 2000 or more dimethylsiloxane repeating units.

In an aspect of the second embodiment, the polymeric material comprises about 50 or more polyethylene glycol-substituted dimethylsiloxane repeating units.

In an aspect of the second embodiment, a number ratio of repeating units derived from an unsubstituted cyclosiloxane monomer to repeating units derived from a cyclosiloxane monomer substituted with a hydrophile is from about 80:1 to about 20:1.

In an aspect of the second embodiment, a number ratio of repeating units derived from an unsubstituted cyclosiloxane monomer to repeating units derived from a cyclosiloxane monomer substituted with a hydrophile is from about 50:1 to about 30:1.

In an aspect of the second embodiment, a number ratio of repeating units derived from an unsubstituted cyclosiloxane monomer to repeating units derived from a cyclosiloxane monomer substituted with a hydrophile is about 40:1.

In an aspect of the second embodiment, a number ratio of repeating units derived from an unsubstituted cyclosiloxane monomer to repeating units derived from a cyclosiloxane monomer substituted with diethylene glycol is from about 80:1 to about 20:1.

In an aspect of the second embodiment, a number ratio of repeating units derived from an unsubstituted cyclosiloxane monomer to repeating units derived from a cyclosiloxane monomer substituted with diethylene glycol is from about 50:1 to about 30:1.

In an aspect of the second embodiment, a number ratio of repeating units derived from an unsubstituted cyclosiloxane monomer to repeating units derived from a cyclosiloxane monomer substituted with diethylene glycol is about 40:1.

In an aspect of the second embodiment, a number ratio of repeating units derived from an unsubstituted cyclosiloxane monomer to repeating units derived from a cyclosiloxane monomer substituted with triethylene glycol is from about 80:1 to about 20:1.

In an aspect of the second embodiment, a number ratio of repeating units derived from an unsubstituted cyclosiloxane monomer to repeating units derived from a cyclosiloxane monomer substituted with triethylene glycol is from about 50:1 to about 30:1.

In an aspect of the second embodiment, a number ratio of repeating units derived from an unsubstituted cyclosiloxane monomer to repeating units derived from a cyclosiloxane monomer substituted with triethylene glycol is about 40:1.

In an aspect of the second embodiment, a number ratio of repeating units derived from an unsubstituted cyclosiloxane monomer to repeating units derived from a cyclosiloxane monomer substituted with tetraethylene glycol is from about 80:1 to about 20:1.

In an aspect of the second embodiment, a number ratio of repeating units derived from an unsubstituted cyclosiloxane monomer to repeating units derived from a cyclosiloxane monomer substituted with tetraethylene glycol is from about 50:1 to about 30:1.

In an aspect of the second embodiment, a number ratio of repeating units derived from an unsubstituted cyclosiloxane monomer to repeating units derived from a cyclosiloxane monomer substituted with tetraethylene glycol is about 40:1.

In an aspect of the second embodiment, a number ratio of repeating units derived from an unsubstituted cyclosiloxane monomer to repeating units derived from a cyclosiloxane monomer substituted with polyethylene glycol is from about 80:1 to about 20:1.

In an aspect of the second embodiment, a number ratio of repeating units derived from an unsubstituted cyclosiloxane monomer to repeating units derived from a cyclosiloxane monomer substituted with polyethylene glycol is from about 50:1 to about 30:1.

In an aspect of the second embodiment, a number ratio of repeating units derived from an unsubstituted cyclosiloxane monomer to repeating units derived from a cyclosiloxane monomer substituted with polyethylene glycol is about 40:1.

In a third embodiment, a biocompatible membrane is provided comprising a polymeric material formed from a cyclosiloxane monomer substituted with a hydrophile, an unsubstituted cyclosiloxane monomer, and a polysiloxane monomer terminated with a telechelic group.

In a fourth embodiment, a polymeric material is provided, wherein the polymeric material comprises a repeating unit derived from a polyethyleneglycol-substituted octamethylcyclotetrasiloxane monomer, a repeating unit derived from an unsubstituted octamethylcyclotetrasiloxane monomer, and a repeating unit derived from a vinyldimethylsilyl-terminated polydimethylsiloxane monomer.

In an aspect of the fourth embodiment, the vinyldimethylsilyl-terminated polydimethylsiloxane monomer contributes about 100 or more dimethylsiloxane repeating units to the polymeric material.

In an aspect of the fourth embodiment, the polymeric material comprises about 2000 or more dimethylsiloxane repeating units.

In an aspect of the fourth embodiment, the polymeric material comprises about 50 or more polyethylene glycol-substituted dimethylsiloxane repeating units.

In an aspect of the fourth embodiment, a number ratio of dimethylsiloxane repeating units to polyethylene glycol-substituted dimethylsiloxane repeating units is from about 80:1 to about 20:1.

In an aspect of the fourth embodiment, a number ratio of dimethylsiloxane repeating units to polyethylene glycol-substituted dimethylsiloxane repeating units is from about 50:1 to about 30:1.

In an aspect of the fourth embodiment, a number ratio of dimethylsiloxane repeating units to polyethylene glycol-substituted dimethylsiloxane repeating units is about 40:1.

In a fifth embodiment, a process for preparing a polymeric material for use in fabricating a biocompatible membrane is provided, the process comprising the steps of: providing a first monomer comprising a cyclosiloxane monomer substituted with a hydrophile; providing a second monomer comprising an unsubstituted cyclosiloxane monomer; providing a third monomer comprising a polysiloxane monomer terminated with a telechelic group; providing a polymerization catalyst; and polymerizing the monomers, whereby a polymeric material suitable for use in fabricating a membrane is obtained.

In an aspect of the fifth embodiment, a molar ratio of the second monomer to the first monomer is from about 80:1 to about 20:1.

In an aspect of the fifth embodiment, a molar ratio of the second monomer to the first monomer is from about is from about 50:1 to about 30:1.

In an aspect of the fifth embodiment, a molar ratio of the second monomer to the first monomer is about 40:1.

In a sixth embodiment, a polymeric material is provided, the material comprising a copolymer of Formula A: 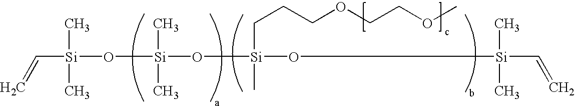
wherein a is an integer of from 100 to 10000; b is an integer of from 1 to 1000; and c is an integer of from 1 to 30.

In an aspect of the sixth embodiment, a ratio of b to a is from about 1:200 to about 1:1.

In an aspect of the sixth embodiment, a ratio of b to a is from about 1:200 to about 1:2.

In an aspect of the sixth embodiment, a ratio of b to a is about 1:200 to about 1:10.

In a seventh embodiment, a process for preparing a polymeric material for use in fabricating a biocompatible membrane is provided, the process comprising the steps of providing a first monomer comprising the Formula B: 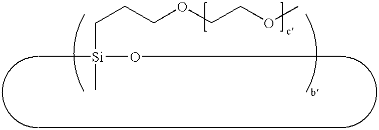
wherein b′ is an integer of from 3 to 6 and c′ is an integer of from 1 to 30; and providing a second monomer comprising the Formula C: 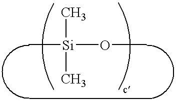
wherein c′ is an integer of from 3 to 6; providing a third monomer comprising the Formula D: 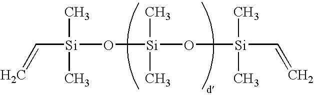
wherein d′ is an integer of from 0 to 100; providing a polymerization catalyst; and polymerizing the monomers, whereby a polymeric material suitable for use in fabricating a membrane is obtained.

In an aspect of the seventh embodiment, a molar ratio of the second monomer to the first monomer is from about 80:1 to about 20:1.

In an aspect of the seventh embodiment, a molar ratio of the second monomer to the first monomer is from about is from about 50:1 to about 30:1.

In an aspect of the seventh embodiment, a molar ratio of the second monomer to the first monomer is about 40:1.

In an eighth embodiment, a polymeric material is provided, wherein the polymeric material comprises a repeating unit derived from a hydrophilically-substituted cyclosiloxane monomer, a repeating unit derived from an unsubstituted cyclosiloxane monomer, and a terminating unit derived from a telechelic siloxane monomer.

In an aspect of the eighth embodiment, the hydrophilically-substituted cyclosiloxane monomer comprises a diethyleneglycol group.

In an aspect of the eighth embodiment, the hydrophilically-substituted cyclosiloxane monomer comprises a triethyleneglycol group.

In an aspect of the eighth embodiment, the hydrophilically-substituted cyclosiloxane monomer comprises a tetraethyleneglycol group.

In an aspect of the eighth embodiment, the hydrophilically-substituted cyclosiloxane monomer comprises a polyethyleneglycol group.

In an aspect of the eighth embodiment, the polyethyleneglycol group comprises an average molecular weight of from about 200 to about 1200.

In an aspect of the eighth embodiment, the hydrophilically-substituted cyclosiloxane monomer comprises a ring size of from about 6 to about 12 atoms.

In an aspect of the eighth embodiment, the unsubstituted cyclosiloxane monomer comprises hexamethylcyclotrisiloxane.

In an aspect of the eighth embodiment, the unsubstituted cyclosiloxane monomer comprises octamethlcyclotetrasiloxane.

In an aspect of the eighth embodiment, the telechelic siloxane monomer comprises divinyltetramethyldisiloxane.

In an aspect of the eighth embodiment, the telechelic siloxane monomer comprises vinyldimethylsilyl terminated polydimethylsiloxane.

In an aspect of the eighth embodiment, the vinyldimethylsilyl terminated polydimethylsiloxane comprises an average molecular weight of from about 200 to about 20000.

In an aspect of the eighth embodiment, the polymeric material comprises about 100 or more dimethylsiloxane repeating units.

In an aspect of the eighth embodiment, the polymeric material comprises from about 100 to about 10000 dimethylsiloxane repeating units.

In an aspect of the eighth embodiment, the polymeric material comprises one or more hydrophilically-substituted repeating units.

In an aspect of the eighth embodiment, the polymeric material comprises from about 1 to about 10000 hydrophilically-substituted repeating units.

In an aspect of the eighth embodiment, the polymeric material comprises one or more polyethylene glycol-substituted repeating units.

In an aspect of the eighth embodiment, the polymeric material comprises from about 1 to about 10000 polyethylene glycol-substituted repeating units.

In an aspect of the eighth embodiment, the polyethyleneglycol comprises an average molecular weight of from about 200 to about 1200.

In an aspect of the eighth embodiment, a number ratio of hydrophilically-substituted siloxane repeating units to unsubstituted siloxane repeating units is from about 1:200 to about 1:1.

In an aspect of the eighth embodiment, a number ratio of hydrophilically-substituted siloxane repeating units to unsubstituted siloxane repeating units is from about 1:200 to about 1:2.

In an aspect of the eighth embodiment, a number ratio of hydrophilically-substituted siloxane repeating units to unsubstituted siloxane repeating units is from about 1:200 to about 1:10.

In an aspect of the eighth embodiment, the polymeric material comprises one or more ethylene glycol-substituted repeating units.

In an aspect of the eighth embodiment, the polymeric material comprises one or more diethylene glycol-substituted repeating units.

In an aspect of the eighth embodiment, the polymeric material comprises one or more triethylene glycol-substituted repeating units.

In an aspect of the eighth embodiment, the polymeric material comprises one or more tetrathyleneglycol-substituted repeating units.

In a ninth embodiment, a method for preparing a biocompatible membrane is provided, the method comprising providing a polymeric material, wherein the polymeric material comprises a repeating unit derived from a cyclosiloxane monomer substituted with a hydrophile, a repeating unit derived from an unsubstituted cyclosiloxane monomer, and a terminating unit derived from a polysiloxane monomer terminated with a telechelic group; mixing the polymeric material with a diluent, whereby a solution or dispersion is obtained; forming the solution or dispersion into a film; and curing the film, wherein the cured film comprises a biocompatible membrane.

In an aspect of the ninth embodiment, the step of forming the solution or dispersion into a film comprises spin coating.

In an aspect of the ninth embodiment, the step of forming the solution or dispersion into a film comprises dip coating.

In an aspect of the ninth embodiment, the step of forming the solution or dispersion into a film comprises casting.

In an aspect of the ninth embodiment, the step of curing comprises curing at elevated temperature.

In an aspect of the ninth embodiment, the method further comprises the step of mixing the polymeric material with a filler.

In an aspect of the ninth embodiment, the filler is selected from the group consisting of fumed silica, aluminum oxide, carbon black, titanium dioxide, calcium carbonate, fiberglass, ceramics, mica, microspheres, carbon fibers, kaolin, clay, alumina trihydrate, wollastonite, talc, pyrophyllite, barium sulfate, antimony oxide, magnesium hydroxide, calcium sulfate, feldspar, nepheline syenite, metallic particles, magnetic particles, magnetic fibers, chitin, wood flour, cotton flock, jute, sisal, synthetic silicates, fly ash, diatomaceous earth, bentonite, iron oxide, nylon fibers, polyethylene terephthalate fibers, poly(vinyl alcohol) fibers, poly(vinyl chloride) fibers, and acrylonitrile fibers.

In an aspect of the ninth embodiment, the cyclosiloxane monomer substituted with a hydrophile comprises a diethyleneglycol group.

In an aspect of the ninth embodiment, the cyclosiloxane monomer substituted with a hydrophile comprises a triethyleneglycol group.

In an aspect of the ninth embodiment, the cyclosiloxane monomer substituted with a hydrophile comprises a tetraethyleneglycol group.

In an aspect of the ninth embodiment, the cyclosiloxane monomer substituted with a hydrophile comprises a polyethyleneglycol group.

In an aspect of the ninth embodiment, the polyethyleneglycol comprises an average molecular weight of from about 200 to about 1200.

In an aspect of the ninth embodiment, the cyclosiloxane monomer substituted with a hydrophile comprises a ring size of from about 6 to about 12 atoms.

In an aspect of the ninth embodiment, the unsubstituted cyclosiloxane monomer comprises hexamethylcyclotrisiloxane.

In an aspect of the ninth embodiment, the unsubstituted cyclosiloxane monomer comprises octamethlcyclotetrasiloxane.

In an aspect of the ninth embodiment, the polysiloxane monomer terminated with a telechelic group comprises divinyltetramethyldisiloxane.

In an aspect of the ninth embodiment, the polysiloxane monomer terminated with a telechelic group comprises vinyldimethylsilyl terminated polydimethylsiloxane.

In an aspect of the ninth embodiment, the vinyldimethylsilyl terminated polydimethylsiloxane comprises an average molecular weight of from about 200 to 20,000.

In an aspect of the ninth embodiment, the polymeric material comprises about 100 or more dimethylsiloxane repeating units.

In an aspect of the ninth embodiment, the polymeric material comprises from about 100 to about 10000 dimethylsiloxane repeating units.

In an aspect of the ninth embodiment, the polymer comprises one or more hydrophilically-substituted repeating units.

In an aspect of the ninth embodiment, the polymeric material comprises from about 1 to about 10000 hydrophilically-substituted repeating units.

In an aspect of the ninth embodiment, the polymeric material comprises one or more polyethylene glycol-substituted repeating units.

In an aspect of the ninth embodiment, the polymeric material comprises from about 1 to about 10000 polyethylene glycol-substituted repeating units.

In an aspect of the ninth embodiment, the polyethyleneglycol comprises an average molecular weight of from about 200 to about 1200.

In an aspect of the ninth embodiment, a number ratio of repeating units derived from cyclosiloxane monomer substituted with a hydrophile to repeating units derived from unsubstituted cyclosiloxane in the polymer is from about 1:200 to about 1:1.

In an aspect of the ninth embodiment, a number ratio of repeating units derived from cyclosiloxane monomer substituted with a hydrophile to repeating units derived from unsubstituted cyclosiloxane in the polymer is from about 1:200 to about 1:2.

In an aspect of the ninth embodiment, a number ratio of repeating units derived from cyclosiloxane monomer substituted with a hydrophile to repeating units derived from unsubstituted cyclosiloxane in the polymer is from about 1:200 to about 1:10.

In an aspect of the ninth embodiment, the polymeric material comprises one or more ethylene glycol-substituted repeating units.

In an aspect of the ninth embodiment, the polymeric material comprises one or more diethylene glycol-substituted repeating units.

In an aspect of the ninth embodiment, the polymeric material comprises one or more triethylene glycol-substituted repeating units.

In an aspect of the ninth embodiment, the polymeric material comprises one or more tetrathyleneglycol-substituted repeating units.

### BRIEF DESCRIPTION OF THE DRAWINGS

FIG. 1 is an exploded perspective view of a glucose sensor incorporating a biocompatible membrane of a preferred embodiment.

FIG. 2 is a graph that shows a raw data stream obtained from a glucose sensor over a 36 hour time span in one example.

FIG. 3 is an illustration of the biocompatible membrane of the device of FIG. 1.

FIG. 4A is a schematic diagram of oxygen concentration profiles through a prior art membrane.

FIG. 4B is a schematic diagram of oxygen concentration profiles through the biocompatible membrane of the preferred embodiments.

FIG. 5 is a Fourier-Transform InfraRed spectrum of Compound I.

FIG. 6 is a Fourier-Transform InfraRed spectrum of Copolymer II.

FIG. 7 is a graph that illustrates percentage of functional sensors at various oxygen concentrations.

### DETAILED DESCRIPTION OF THE PREFERRED EMBODIMENT

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

### DEFINITIONS

In order to facilitate an understanding of the preferred embodiments, terms as employed herein are defined as follows.

Herein, the values for the variables in the formulas are integers; however, they can be average values if the formulas represent average structures, such as occur with polymers.

As used herein, the term “copolymer” is a broad term and is used in its ordinary sense, including, without limitation, polymers having two, three, four, or more different repeat units and includes copolymers, terpolymers, tetrapolymers, and the like.

As used herein, the term “telechelic” is a broad term and is used in its ordinary sense, including, without limitation, to refer to polymers designed to contain terminal functional groups.

As used herein, the term “organic group” is a broad term and is used in its ordinary sense, including, without limitation, a hydrocarbon group that can be classified as an aliphatic group, cyclic group, or combination of aliphatic and cyclic groups (for example, alkaryl and aralkyl groups). In the context of the preferred embodiments, the term “aliphatic group” refers to a saturated or unsaturated linear or branched hydrocarbon group. This term encompasses alkyl, alkenyl, and alkynyl groups. The term “alkyl group” refers to a saturated linear or branched hydrocarbon group including, for example, methyl, ethyl, isopropyl, t-butyl, heptyl, dodecyl, octadecyl, amyl, 2-ethylhexyl, and the like. The term “alkenyl group” refers to an unsaturated, linear or branched hydrocarbon group with one or more carbon-carbon double bonds, such as a vinyl group. The term “alkynyl group” refers to an unsaturated, linear or branched hydrocarbon group with one or more carbon-carbon triple bonds. The term “cyclic group” refers to a closed ring hydrocarbon group that is classified as an alicyclic group, aromatic group, or heterocyclic group. The term “alicyclic group” refers to a cyclic hydrocarbon group having properties resembling those of aliphatic groups. The term “aromatic group” or “aryl group” refers to a mononuclear or polynuclear aromatic hydrocarbon group. The term “heterocyclic group” refers to a closed ring hydrocarbon group, either aromatic or aliphatic, in which one or more of the atoms in the ring is an element other than carbon (including but not limited to nitrogen, oxygen, and sulfur).

As is well understood in this technical area, a large degree of substitution on organic groups is not only tolerated, but is often advisable. The compounds of the preferred embodiments include both substituted and unsubstituted organic groups. To simplify the discussion and recitation of certain terminology used herein, the terms “group” and “moiety” are employed to differentiate between chemical species that allow for substitution or that may be substituted and those that do not allow or may not be so substituted. Thus, when the term “group” is used to describe a chemical substituent, the described chemical material includes the unsubstituted group and that group with O, N, or S atoms, for example, in the chain as well as carbonyl groups or other conventional substituents. Where the term “moiety” is employed to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, and the like. Thus, “alkyl group” includes ether groups, haloalkyls, nitroalkyls, carboxyalkyls, hydroxyalkyls, sulfoalkyls, and the like. On the other hand, the phrase “alkyl moiety” is limited to the inclusion of only pure open chain saturated hydrocarbon alkyl substituents, such as methyl, ethyl, propyl, t-butyl, and the like.

The term “analyte” as used herein is a broad term and is used in its ordinary sense, including, without limitation, a substance or chemical constituent in a biological fluid (for example, blood, interstitial fluid, cerebral spinal fluid, lymph fluid or urine) that can be analyzed. Analytes may include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobinopathies, A,S,C,E, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17 alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins and hormones naturally occurring in blood or interstitial fluids may also constitute analytes in certain embodiments. The analyte may be naturally present in the biological fluid, for example, a metabolic product, a hormone, an antigen, an antibody, and the like. Alternatively, the analyte may be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body may also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

The term “sensor” as used herein is a broad term and is used in its ordinary sense, including, without limitation, the component or region of a device by which an analyte can be quantified.

The terms “operably connected” and “operably linked” as used herein are broad terms and are used in their ordinary sense, including, without limitation, one or more components being linked to another component(s) in a manner that allows transmission of signals between the components, for example, wired or wirelessly. For example, one or more electrodes may be used to detect the amount of analyte in a sample and convert that information into a signal; the signal may then be transmitted to an electronic circuitry. In this case, the electrode is “operably linked” to the electronic circuitry.

The terms “raw data stream” and “data stream,” as used herein, are broad terms and are used in their ordinary sense, including, without limitation, an analog or digital signal directly related to the measured glucose from a glucose sensor. In one example, the raw data stream is digital data in “counts” converted by an A/D converter from an analog signal (e.g., voltage or amps) representative of a glucose concentration. The terms broadly encompass a plurality of time spaced data points from a substantially continuous glucose sensor, which comprises individual measurements taken at time intervals ranging from fractions of a second up to, e.g., 1, 2, or 5 minutes or longer.

The term “counts,” as used herein, is a broad term and is used in its ordinary sense, including, without limitation, a unit of measurement of a digital signal. In one example, a raw data stream measured in counts is directly related to a voltage (e.g., converted by an A/D converter), which is directly related to current from the working electrode. In another example, counter electrode voltage measured in counts is directly related to a voltage.

The term “host” as used herein is a broad term and is used in its ordinary sense, including, without limitation, mammals, particularly humans.

The terms “foreign body response,” “FBR,” “foreign body capsule,” and “FBC” as used herein are broad terms and used in their ordinary sense, including, without limitation, body's response to the introduction of a foreign object, which forms a capsule around the foreign object. There are three main layers of a foreign body capsule (FBC): the innermost layer, adjacent to the object, is composed generally of macrophages, foreign body giant cells, and occlusive cell layers; the intermediate FBC layer, lying distal to the first layer with respect to the object, is a wide zone (for example, about 30-100 microns) composed primarily of fibroblasts, contractile fibrous tissue fibrous matrix; and the outermost FBC layer is loose connective granular tissue containing new blood vessels. Over time, this FBC tissue becomes muscular in nature and contracts around the foreign object so that the object remains tightly encapsulated.

The term “barrier cell layer” as used herein is a broad term and is used in its ordinary sense, including, without limitation, a cohesive monolayer of cells (for example, macrophages and foreign body giant cells) that substantially blocks the transport of molecules across the a surface that is exposed to the host's bodily fluid.

The term “cellular attachment” as used herein is a broad term and is used in its ordinary sense, including, without limitation, adhesion of cells and/or cell processes to a material at the molecular level, and/or attachment of cells and/or cell processes to micro- (or macro-) porous material surfaces. One example of a material used in the prior art that allows cellular attachment due to porous surfaces is the BIOPORE™ cell culture support marketed by Millipore (Bedford, Mass.).

The term “cell processes” as used herein is a broad term and is used in its ordinary sense, including, without limitation, pseudopodia of a cell.

The term “domain” as used herein is a broad term and is used in its ordinary sense, including, without limitation, regions of the biocompatible membrane that may be layers, uniform or non-uniform gradients (for example, anisotropic), functional aspects of a material, or provided as portions of the membrane.

The term “solid portions” as used herein is a broad term and is used in its ordinary sense, including, without limitation, a solid material having a mechanical structure that demarcates cavities, voids, or other non-solid portions.

The term “substantial” as used herein is a broad term and is used in its ordinary sense, including, without limitation, an amount greater than 50 percent.

The term “co-continuous” as used herein is a broad term and is used in its ordinary sense, including, without limitation, a solid portion wherein an unbroken curved line in three dimensions exists between any two points of the solid portion.

The phrase “distal to” refers to the spatial relationship between various elements in comparison to a particular point of reference. For example, some embodiments of a device include a biocompatible membrane having a cell disruptive domain and a cell impermeable domain. If the sensor is deemed to be the point of reference and the cell disruptive domain is positioned farther from the sensor, then that domain is distal to the sensor.

The term “proximal to” refers to the spatial relationship between various elements in comparison to a particular point of reference. For example, some embodiments of a device include a biocompatible membrane having a cell disruptive domain and a cell impermeable domain. If the sensor is deemed to be the point of reference and the cell impermeable domain is positioned nearer to the sensor, then that domain is proximal to the sensor.

The term “hydrophile” and “hydrophilic” as used herein are broad terms and are used in their ordinary sense, including, without limitation, a chemical group that has a strong affinity for water. Representative hydrophilic groups include but are not limited to hydroxyl, amino, amido, imido, carboxyl, sulfonate, alkoxy, ionic, and other groups.

The term “hydrophile-substituted” and “hydrophilically-substituted” as used herein are broad terms and are used in their ordinary sense, including, without limitation, a polymer or molecule that includes as a substituent a chemical group that has a strong affinity for water.

The term “hydrophobically-substituted siloxane repeating unit” as used herein is a broad term and is used in its ordinary sense, including, without limitation, a siloxane repeating unit that has been subjected to grafting or substitution with a hydrophobe.

The term “hydrophilically-substituted siloxane repeating unit” as used herein is a broad term and is used in its ordinary sense, including, without limitation, a siloxane repeating unit that has been subjected to grafting or substitution with a hydrophile.

The term “hydrophobe” and “hydrophobic” as used herein are broad terms and are used in their ordinary sense, including, without limitation, a chemical group that does not readily absorb water, is adversely affected by water, or is insoluble in water.

The term “covalently incorporated” as used herein is a broad term and is used in its ordinary sense, including, without limitation, a chemical bond in which the attractive force between atoms is created by the sharing of electrons.

The term “grafting” as used herein is a broad term and is used in its ordinary sense, including, without limitation, a polymer reaction in which a chemical group is attached to a polymer molecule having a constitutional or configurational feature different from that of the attached group. Grafting can include, but is not limited to attaching one or more side chains to a polymeric backbone.

The term “FTIR” as used herein is a broad term and is used in its ordinary sense, including, without limitation, Fourier-Transform Infrared Spectroscopy (FTIR). FTIR is a technique wherein a sample is subjected to excitation of molecular bonds by infrared radiation and measurement of the absorption spectrum for chemical bond identification in organic and some inorganic compounds.

The term “silicone composition” as used herein is a broad term and is used in its ordinary sense, including, without limitation, a composition of matter that comprises polymers having alternating silicon and oxygen atoms in the backbone.

The term “oxygen antenna domain” as used herein is a broad term and is used in its ordinary sense, including, without limitation, a domain composed of a material that has higher oxygen solubility than aqueous media so that it concentrates oxygen from the biological fluid surrounding the biocompatible membrane. In one embodiment, the properties of silicone (and/or silicone compositions) inherently enable domains formed from silicone to act as an oxygen antenna domain. The characteristics of an oxygen antenna domain enhance function in a glucose sensor by applying a higher flux of oxygen to certain locations.

Overview

Biocompatible membranes and implantable devices incorporating such biocompatible membranes in are provided herein. For example, the biocompatible membranes of preferred embodiments can be utilized with implantable devices and methods for monitoring and determining analyte levels in a biological fluid, such as for measuring glucose levels of individuals having diabetes.

Although many of the preferred embodiments are directed at analyte sensors including the preferred biocompatible membranes and methods for their use, these biocompatible membranes are not limited to use in devices that measure or monitor analytes (including, but not limited to, glucose, cholesterol, amino acids, lactate, and the like). Rather, these biocompatible membranes may be employed in a variety of devices that are concerned with the controlled transport of biological fluids, especially those involving measurement of analytes that are substrates for oxidase enzymes (see, e.g., U.S. Pat. No. 4,703,756), cell transplantation devices (see, e.g., U.S. Pat. Nos. 6,015,572, 5,964,745, and 6,083,523), electrical delivery and/or measuring devices such as implantable pulse generation cardiac pacing devices (see, e.g., U.S. Pat. Nos. 6,157,860, 5,782,880, and 5,207,218), electrocardiogram device (see, e.g., U.S. Pat. Nos. 4,625,730 and 5,987,352), and electrical nerve stimulating devices (see, e.g., U.S. Pat. Nos. 6,175,767, 6,055,456, and 4,940,065). Other examples include utilizing the biocompatible membranes for transplanted cells, for example, transplanted genetic engineered cells, Islets of Langerhans (either allo, auto or xeno type) as pancreatic beta cells to increase the diffusion of nutrients to the islets, as well utilizing the membranes in a biosensor to sense glucose in the tissues of the patient so as to monitor the viability of the implanted cells.

Implantable devices for determining analyte concentrations in a biological system can utilize the biocompatible membranes of the preferred embodiments to selectively permit the passage of analytes, thereby assuring accurate measurement of the analyte in vivo, such as described herein. Cell transplantation devices can utilize the biocompatible membranes of the preferred embodiments to protect the transplanted cells from attack by host inflammatory or immune response cells while simultaneously allowing nutrients as well as other biologically active molecules needed by the cells for survival.

The materials contemplated for use in preparing the biocompatible membranes also result in membranes wherein biodegradation is eliminated or significantly delayed, which can be desirable in devices that continuously measure analyte concentrations or deliver drugs, or in cell transplantation devices. For example, in a glucose-measuring device the electrode surfaces of the glucose sensor are in contact with (or operably connected with) a thin electrolyte phase, which in turn is covered by a membrane that contains an enzyme, for example, glucose oxidase, and a polymer system, such as described in U.S. Published Patent Application 2003/0032874. In this example, the biocompatible membrane covers the enzyme membrane and serves, at least in part, to protect the sensor from external forces and factors that may result in biodegradation. By significantly delaying biodegradation of the sensor, accurate data may be collected over long periods of time (for example, months to years). Similarly, biodegradation of the biocompatible membrane of implantable cell transplantation devices can allow host inflammatory and immune cells to enter the device, thereby compromising long-term function.

Silicones

Silicones (for example, organosiloxanes) are polymers containing alternating silicon and oxygen atoms in the backbone and having various organic groups attached to the silicon atoms of the backbone. Silicone copolymers include backbone units that possess a variety of groups attached to the silicone atoms. Both silicones and silicone copolymers are useful materials for a wide variety of applications (for example, rubbers, adhesives, sealing agents, release coatings, antifoam agents). Because of their biocompatibility, silicones present a low risk of unfavorable biological reactions and have therefore gained the medical industry's recognition as being useful in a wide variety of medical devices. However, silicone is an inherently hydrophobic material, and therefore does not permit the transport of glucose and other such water-soluble molecules (for example, drugs). Thus, silicone membranes have not previously been simply and reliably implemented in analyte sensors.

It is noted that in general, conventional hydrophilic silicone compositions that possess grafted hydrophilic groups have a molecular weight between about 200 and about 50,000 g/mol. This molecular weight is typically chosen to provide properties desirable for cosmetic products. For example, silicones may be employed as plasticizing resins in hair spray and gel products without diminishing hold. Silicones impart improved skin feel, wet and dry compatibility, conditioning of hair, and replacement of lipids and natural oils on the skin surface. The molecular weights for such materials are typically low, for example, below 50,000 g/mol, so as to provide the above-described properties in cosmetic formulations. However, silicone compositions with the above-described conventional molecular weight would not facilitate the preparation of cross-linked membranes that provide the strength and toughness useful in the preferred embodiments; they typically do not possess functionality, for example telechelic character, which allows further chemical cross-linking of the composition. In contrast to conventional silicone compositions, the preferred embodiments provide a silicone composition that has a molecular weight between about 50,000 to about 800,000 g/mol, which possesses functionality, for example functional endgroups, which facilitates fabrication of cross-linked membranes. Polymers of the preferred embodiments formed with this molecular weight range facilitate the preparation of cross-linked biocompatible membranes that provide the strength, tear resistance, stability, and toughness advantageous for use in vivo.

The Polymerization Reaction

The preferred embodiments provide cyclic siloxane monomers that are substituted with a hydrophilic group. These hydrophile-grafted monomers are preferably polymerized using ring-opening polymerization, either alone or in the presence of cyclic siloxane monomers, to yield random and block siloxane copolymers. This methodology facilitates a high degree of polymerization since the hydrophile-grafted cyclic siloxane monomers can be easily purified and the ring opening polymerization is an efficient reaction. Alternatively, the polymers of the preferred embodiments can be prepared by coequilibrating mixtures of cyclic and linear species.

The copolymerization reactions preferably utilize similar chemistries as are known in the art of preparing silicone materials so as to yield copolymers having various functionalities either pendant and/or terminal to the polymer backbone. Pendant and/or terminally functional hydrophile-grafted copolymers can be employed as elastomers, adhesives, and sealing agents. Such copolymers are capable of being crosslinked. The crosslinked materials can be suitable for a variety of applications, including but not limited to elastomers, adhesives, sealing agents, and the like. They are particularly suitable for use in medical devices.

The Monomers

In a preferred embodiment, hydrophile-grafted cyclic siloxane monomers having the following Formula (a) are provided: 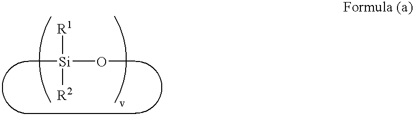
wherein v is at least 3, R¹ is a hydrophile group, and R² is a monovalent organic group.

In another preferred embodiment, asymmetric cyclic hydrophile-grafted cyclic siloxane monomers having the following Formula (b) are provided: 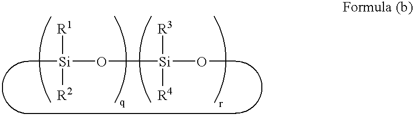
wherein q and r are each at least 1, with the proviso that the sum of q and r is at least 3, R¹ is a hydrophile group and each R², R³, and R⁴, which can be the same or different, is a monovalent organic group.

The Polymerization Initiators or Catalysts

The cyclic hydrophile-grafted siloxane monomers can be polymerized using methods that are similar to those preferred for preparing other siloxanes because the monomer backbone still consists of alternating silicon and oxygen atoms. For example, depending upon the ring size, the cyclic hydrophile-grafted monomers can undergo ring-opening reactions under either anionic or cationic catalysis. The anionic polymerization of cyclic hydrophile-grafted monomers can be initiated by alkali metal oxides and hydroxides, silanolates and other bases. Preferably, anionic polymerization is conducted in potassium trimethylsilanoate and phosphazene base, P₄-t-bu, solution. Alternatively, cationic polymerization can be initiated by protonic and Lewis acids, preferably triflic acid or strongly acidic ion-exchange resins.

Typically, both anionic and cationic ring opening polymerizations (ROP) may be performed without the use of solvents. However, in order to deliver well-controlled amounts of catalyst to reaction mixtures, solvents such as toluene or hexanes may be employed as diluents for the catalyst. Both the anionic and cationic catalyzed equilibration reaction conditions (for example, time and temperature) are similar to those known in the art for ROP of cyclic organosiloxanes. Once added to the cyclic monomer mixture, the equilibration reaction can typically be completed within about 30 minutes to several hours.

Siloxane Copolymers

Hydrophile-grafted siloxane copolymers of the following Formula (c) are also provided: 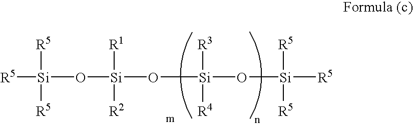
wherein m and n are at least 1, with the proviso that the sum of m and n is at least about 300, R¹ is a hydrophile group and each R², R³R⁴, and R⁵, which can be the same or different, is a monovalent organic group. In preferred embodiments, n is preferably from about 1 to about 1000 or more, more preferably from about 1, 2, 3, 4, 5, 6, 7, 9, or 10 to about 400, 450, 500, 550, 600, 650, 700, 750, 800, 850, 900 or 950, and most preferably from about 20, 30, 40, 50, 60, 70, 80, or 90 to about 100, 125, 150, 175, 200, 225, 250, 275, 350, or 375. In preferred embodiments, m is preferably from about 1 to about 1000, 2000, 3000, 4000, 5000, 6000, 7000, 8000, 9000, or 10000 or more, more preferably from about 1, 2, 3, 4, 5, 6, 7, 9, or 10 to about 400, 450, 500, 550, 600, 650, 700, 750, 800, 850, 900 or 950, and most preferably from about 20, 30, 40, 50, 60, 70, 80, or 90 to about 100, 125, 150, 175, 200, 225, 250, 275, 350, or 375. The ratio of m:n is preferably from about 1:200 or higher to about 1:1 or lower, more preferably from about 1:200, 1:175, 1:150, 1:125, 1:100, 1:90, 1:80, 1:70, 1:60, 1:50, 1:40, 1:30, or 1:20 to about 1:2, and most preferably from about 1:20, 1:19, 1:18, 1:17, 1:16, 1:15, 1:14, 1:13, 1:12, or 1:11 to about 1:3, 1:4, 1:5, 1:6, 1:7, 1:8, 1:9, or 1:10.

Cyclic hydrophile-grafted monomers (including mixtures of symmetric and asymmetric cyclic monomers) can be copolymerized in the presence of cyclic and/or linear siloxane compounds according to the methods of preferred embodiments. A representative synthesis of such copolymers is described, for example, by the following scheme (Scheme 1): 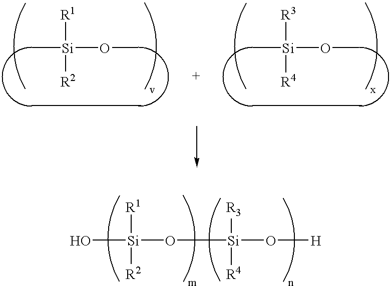
wherein R¹, R², R³, R⁴, R⁵, v, x, m, and n are as defined above. The value of v and x is at least 3. In preferred embodiments, m is preferably from about 1 to about 1000 or more, more preferably from about 1, 2, 3, 4, 5, 6, 7, 9, or 10 to about 400, 450, 500, 550, 600, 650, 700, 750, 800, 850, 900 or 950, and most preferably from about 20, 30, 40, 50, 60, 70, 80, or 90 to about 100, 125, 150, 175, 200, 225, 250, 275, 350, or 375. In preferred embodiments, n is preferably from about 1 to about 1000, 2000, 3000, 4000, 5000, 6000, 7000, 8000, 9000, or 10000 or more, more preferably from about 1, 2, 3, 4, 5, 6, 7, 9, or 10 to about 400, 450, 500, 550, 600, 650, 700, 750, 800, 850, 900 or 950, and most preferably from about 20, 30, 40, 50, 60, 70, 80, or 90 to about 100, 125, 150, 175, 200, 225, 250, 275, 350, or 375. The ratio of m:n is preferably from about 1:200 or higher to about 1:1 or lower, more preferably from about 1:200, 1:175, 1:150, 1:125, 1:100, 1:90, 1:80, 1:70, 1:60, 1:50, 1:40, 1:30, or 1:20 to about 1:2, and most preferably from about 1:20, 1:19, 1:18, 1:17, 1:16, 1:15, 1:14, 1:13, 1:12, or 1:11 to about 1:3, 1:4, 1:5, 1:6, 1:7, 1:8, 1:9, or 1:10. Each R², R³ and R⁴ group, which can be the same or different, is preferably, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₁, C₂₂, C₂₃, C₂₄, C₂₅, C₂₆, C₂₇, C₂₈, C₂₉, or C₃₀ organic group. Preferably, R², R³, and R⁴ are independently selected from methyl, ethyl, propyl, butyl, pentyl, hexyl, or other alkyl groups; vinyl or other alkenyl groups; phenyl, tolyl, xylyl, or other aryl groups; or benzyl, phenethyl, or other aralkyl groups. These groups may be substituted in part or in whole (for example, such that all of the hydrogen atoms are replaced) with various groups, such as, for example, halogen atoms including fluoro, chloro, bromo, and iodo, cyano groups, and amino groups. More preferably, R³ and R⁴ are independently selected from methyl, phenyl, and vinyl moieties. The resultant copolymers can be random or block copolymers, or can have another arrangement of monomers. The structural unit containing R³ and R⁴ groups in the above scheme is referred to as a siloxane unit and the structural unit containing the R¹ and R² groups is referred to as a hydrophile-grafted unit.

Terminal or Pendant Groups

Hydrophile-grafted siloxane copolymers containing terminal and/or pendant functional groups can be produced, for example, according to the following scheme (Scheme 2): 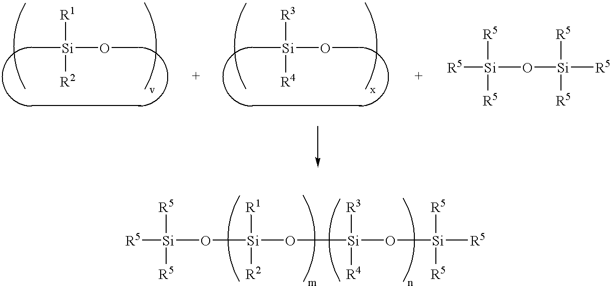
wherein R¹, R², R³, R⁴, v, x, m, and n are as defined above, and wherein each R⁵ group is independently a monovalent organic group (preferably a C₁ to C₃₀, organic group). Preferably, each R⁵ is independently a methyl, ethyl, propyl, butyl, pentyl, hexyl, or other alkyl group; a vinyl, allyl, or other alkenyl group; a phenyl, tolyl, xylyl, or other aryl group; or a benzyl, phenethyl, or other aralkyl group. These groups may be substituted in part or in whole (namely, such that all the hydrogen atoms are replaced) with various groups, such as, for example, halogen atoms, cyano groups, and amino groups. More preferably, each terminal silyl group includes at least one R⁵, which can be a vinyl moiety. The resulting copolymers can be random, block, tapered, or of another configuration.

Fillers

Reinforcement and enhanced physical properties of membranes made with the copolymers provided herein are obtained when treated fumed silica is compounded with hydrophile-grafted copolymers having pendent functional groups. The preferred functionalized copolymers can be compounded with a silica filler (for example, fumed silica) and/or cross-linked using similar chemistries as are known in the art for silicone rubber. Other fillers suitable for use include but are not limited to aluminum oxide, carbon black, titanium dioxide, calcium carbonate, fiberglass, ceramics, mica, microspheres, carbon fibers, kaolin and other clays, alumina trihydrate, wollastonite, talc, pyrophyllite, barium sulfate, antimony oxide, magnesium hydroxide, calcium sulfate, feldspar, nepheline syenite, metallic and magnetic particles and fibers, natural products such as chitin, wood flour, cotton flock, jute and sisal, synthetic silicates, fly ash, diatomaceous earth, bentonite, iron oxide, and synthetic fibers such as nylon, polyethylene terephthalate, poly(vinyl alcohol), poly(vinyl chloride) and acrylonitrile.

Crosslinking

In certain preferred embodiments, one or more of the R groups (R¹, R², R³, R⁴, and/or R⁵) of the copolymers in the above formulae include crosslinkable functionalities, such as vinyl, alkoxy, acetoxy, enoxy, oxime, amino, hydroxyl, cyano, halo, acrylate, epoxide, isocyanato groups, and the like. In particularly preferred embodiments, copolymers, whether cross-linked or not, are compounded with a silica filler, which typically provides reinforcement and superior physical properties in certain applications. For such materials, the sum of m and n (Degree of polymerization, Dp) is preferably from about 100 or less to about 450, 500, 600, 700, 800, 900, 1000, 2000, 3000, 4000, 5000, 6000, 7000, 8000, 9000, or 10000 or more, and more preferably from about 110, 120, 130, 140, 150, 160, 170, 180, 190, 200, 210, 220, 230, 240, or 250 to about 260, 270, 280, 290, 300, 310, 320, 330, 340, 350, 360, 370, 380, 390, or 400.

Cyclic hydrophile-grafted siloxane monomers can be polymerized using methods that are similar to those preferred for cyclic siloxanes, such as are described above. Alternatively, hydrophile-grafted siloxane copolymers of preferred embodiments can be prepared by coequilibrating mixtures of cyclic and/or linear species. Coequilibrations can be performed under the same anionic or cationic reaction conditions as described herein for ROP of hydrophile-grafted siloxane copolymers. For example, a cyclic hydrophile-grafted siloxane monomer as described in Formula (a) can be equilibrated with a linear siloxane polymer to yield a hydrophile-grafted silicone copolymer. In addition, a cyclic siloxane monomer can be equilibrated with a hydrophile-grafted siloxane copolymer to afford a hydrophile-grafted siloxane copolymer having incorporated additional siloxane units. Alternatively, a linear hydrophile-grafted siloxane copolymer and linear siloxane polymer can be equilibrated together to yield a copolymer that contains a summation of both linear starting reagent units.

In order to prepare crosslinked hydrophile-grafted siloxane materials, it is preferred for the copolymers to be functionalized and miscible with the crosslinker. When the hydrophile content of a hydrophile-grafted siloxane copolymer is greater than about 15% by weight, the copolymer is not miscible with conventional polysiloxane crosslinking materials. However, if both crosslinking functionalities are terminal and/or pendant to a hydrophile-grafted siloxane copolymer, the materials are typically miscible and will react. Hydrophiles suitable for grafting include but are not limited to mono-, di-, tri- and tetra-ethylene oxides; polyethylene glycol dimethyl ethers such as those of molecular weight 250, 500, 1000, and 2000; polyethylene glycol dibutyl ethers; polypropylene glycol dimethyl ethers; polyalkylene glycol allylmethyl ether of molecular weight 250, 350, 500, 1100, and 1000; and mixtures thereof.

Process of Preparing Films or Membranes

Films or membranes of preferred embodiments may generally be prepared according to the following method. One or more polymers are mixed with one or more fillers, optionally at elevated temperature. One or more crosslinkers, chain extenders, and/or catalysts are then added to the mixture of polymer and filler. The resulting mixture is diluted with a suitable diluent (for example, toluene) to a suitable concentration (for example, 10 wt. % solids or less up to 15, 20, 25, 30, 35, 40, 45, 50, 55, 60, 65, 70, 75, 80, 85, or 90 wt. % solids or more). The diluted mixture is then coated onto a nonstick sheeting, such as polyethylene or Teflon sheeting, using a fixed gap (0.001″ or less up to 0.002″, 0.003″, 0.004″, 0.005″, 0.006″, 0.007″, 0.008″, 0.009″, or 0.010″ or more). The film is then cured at elevated temperature. Other methods of forming films as are known in the art may also be employed, such as solid state extrusion, constrained forming processes, thermoforming, compression and transfer molding, injection molding, spin coating, dip coating, and the like.

While it is generally preferred to employ one or more fillers, in certain embodiments no filler can be employed. In such embodiments, the polymer is dissolved or dispersed in a suitable diluent or solvent prior to forming the film.

Analyte Sensor

One aspect of the preferred embodiments relates to biocompatible membranes useful in analyte-measuring devices that measure a concentration of an analyte of interest or a concentration of a substance indicative of the concentration or presence of an analyte (for example, glucose). In certain embodiments, the analyte-measuring device is capable of continuous operation, and can include, for example, a subcutaneous, transdermal, or intravascular device. In some embodiments, the device can analyze a single blood sample. The analyte-measuring device can employ any method of analyte-measurement, including but not limited to one or more of chemical, physical, enzymatic, an/or optical analysis.

The analyte sensor useful with the preferred embodiments can include any device capable of measuring the concentration of an analyte of interest. One exemplary embodiment is described below, which utilizes an implantable glucose sensor. However, it is understood that the devices and methods described herein can be applied to any device capable of measuring a concentration of an analyte and providing an output signal indicative of the concentration of the analyte.

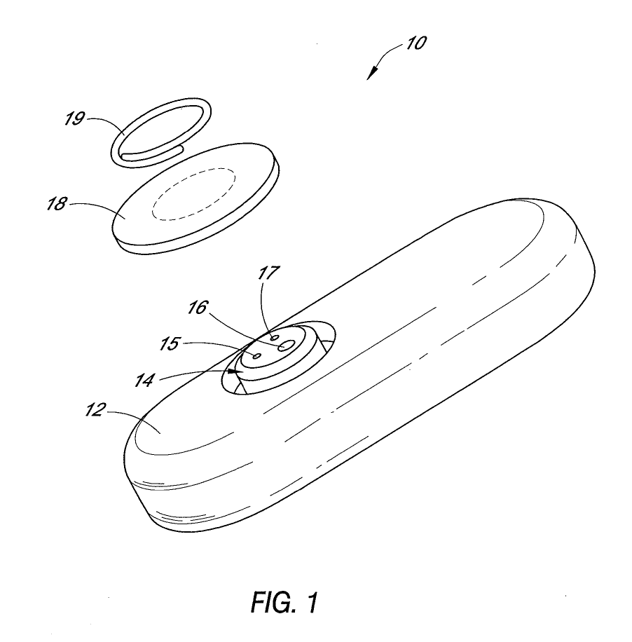

FIG. 1 is an exploded perspective view of an implantable glucose sensor 10 that utilizes amperometric electrochemical sensor technology to measure glucose. In this embodiment, a body 12 and head 14 house the electrodes 15, 16, and 17 and sensor electronics (not shown). The three electrodes are operably connected to the sensor electronics and are covered by a biocompatible membrane 18, which is attached by a clip 19.

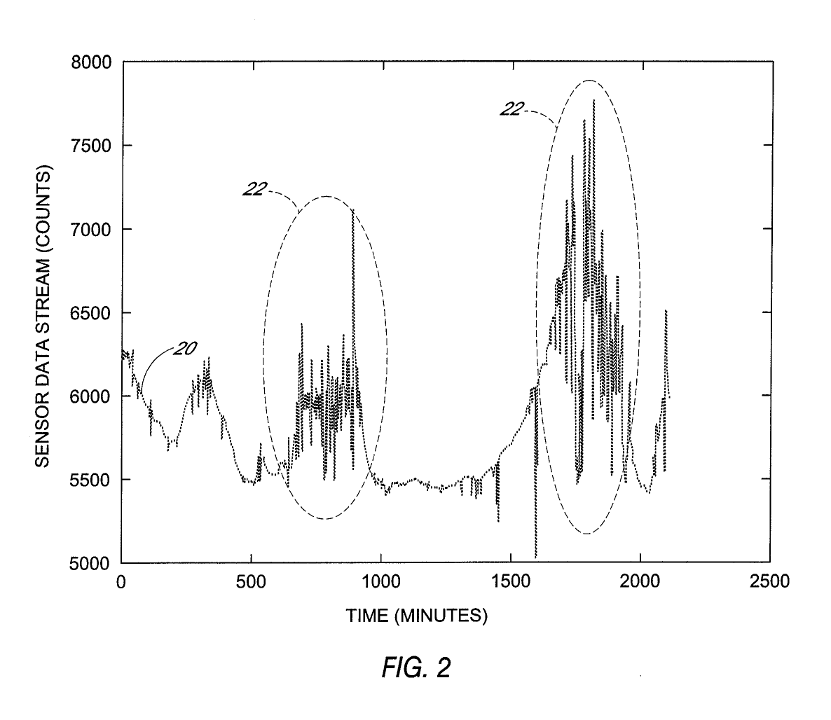

The three electrodes 15, 16, and 17, which extend through the head 14, include a platinum working electrode 15, a platinum counter electrode 16, and a silver/silver chloride reference electrode 17. The top ends of the electrodes comprise active electrochemical surfaces and are in contact with an electrolyte phase (not shown), which is a free-flowing fluid phase disposed between the biocompatible membrane 18 and the electrodes 15, 16, and 17 upon assembly. The biocompatible membrane 18 is described in more detail below with reference to FIG. 2.

In the embodiment depicted in FIG. 1, the counter electrode 16 is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction:
Glucose+O₂→Gluconate+H₂O₂

The change in H₂O₂ can be monitored to determine glucose concentration, in that for each glucose molecule metabolized, there is a proportional change in the product H₂O₂. Oxidation of H₂O₂ by the working electrode is balanced by a reduction of ambient oxygen, enzyme generated H₂O₂, or other reducible species at the counter electrode. The H₂O₂ produced from the glucose oxidase reaction further reacts at the surface of the working electrode and produces two protons (2H⁺), two electrons (2e⁻), and one oxygen molecule (O₂).

In one embodiment, a potentiostat applies a constant potential between the working and reference electrodes to produce a current value. The current that is produced at the working electrode (and flows through the circuitry to the counter electrode) is proportional to the diffusional flux of H₂O₂. Accordingly, a raw signal is produced that is representative of the concentration of glucose in the patient's body, and therefore can be utilized to estimate a meaningful glucose value as described herein.

For a glucose sensor to be useful, glucose is preferably the limiting reagent. Preferably, the oxygen concentration is in excess at all potential glucose concentrations. In electrochemical sensors, there are two main pathways by which oxygen can be consumed at the counter electrode. These pathways include a four-electron pathway to produce hydroxide and a two-electron pathway to produce hydrogen peroxide. In addition to the counter electrode, oxygen is further consumed by the glucose oxidase within the enzyme layer. Therefore, due to the oxygen consumption by both the enzyme and the counter electrode, there is a net consumption of oxygen within the electrode system.

FIG. 2 is a graph that shows a raw data stream obtained from a glucose sensor with a conventional biocompatible membrane. The x-axis represents time in minutes. The y-axis represents sensor data in counts. In this example, sensor output in counts is transmitted every 30-seconds. The raw data stream 20 includes substantially smooth sensor output in some portions, however other portions exhibit transient non-glucose related signal artifacts 22 that have higher amplitude than normal system noise.

While not wishing to be bound by theory, it is believed that conventional subcutaneously implanted sensors undergo transient ischemia that compromises sensor function. Particularly, referring to the signal artifacts 22 in FIG. 2, it is believed that local ischemia creates an enzymatic reaction that is rate-limited by oxygen, which is responsible for non-glucose related decreased sensor output. In this situation, glucose is expected to build up in the membrane because it is not completely catabolized during the oxygen deficit. When oxygen is again in excess, there is also excess glucose due to the transient oxygen deficit. The enzyme rate then speeds up for a short period until the excess glucose is catabolized, resulting in spikes of non-glucose related increased sensor output.

Because excess oxygen (relative to glucose) is necessary for proper sensor function, transient ischemia can result in a loss of signal gain in the sensor data. In some situations, transient ischemia can occur at high glucose levels, wherein oxygen can become limiting to the enzymatic reaction, resulting in a non-glucose dependent downward trend in the data. In some situations, certain movements or postures taken by the patient can cause transient signal artifacts as blood is squeezed out of the capillaries resulting in local ischemia, and causing non-glucose dependent signal artifacts. In some situations, oxygen can also become transiently limited due to contracture of tissues around the sensor interface. This is similar to the blanching of skin that can be observed when one puts pressure on it. Under such pressure, transient ischemia can occur in both the epidermis and subcutaneous tissue. Transient ischemia is common and well tolerated by subcutaneous tissue. However, such ischemic periods can cause an oxygen deficit in implanted sensors that may last for many minutes or even an hour or longer.

In order to overcome the effects of transient ischemia, the biocompatible membranes 18 of the preferred embodiments comprise materials with a high oxygen solubility. These materials act as an oxygen antenna domain providing a reserve of oxygen that may be used to compensate for the local oxygen deficit during times of transient ischemia. As a result, the biocompatible membranes of the preferred embodiments enable glucose sensors and other devices such as drug delivery and cell transplantation devices to function in the subcutaneous space even during local transient ischemia.

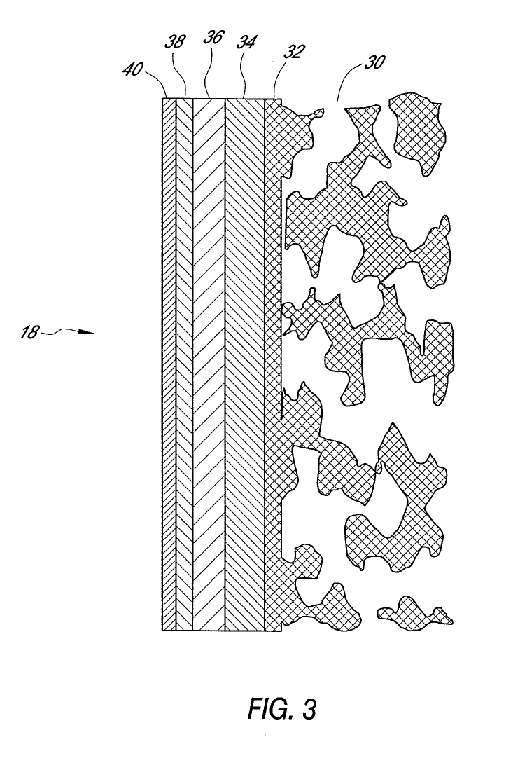

As described below with reference to FIG. 3, the biocompatible membrane 18 can include two or more domains that cover and protect the electrodes of an implantable glucose-measuring device. In such an embodiment, the membrane prevents direct contact of the biological fluid sample with the electrodes, while controlling the permeability of selected substances (for example, oxygen and analytes) present in the biological fluid through the membrane for reaction in an enzyme rich domain with subsequent electrochemical reaction of formed products at the electrodes.

The electrode surfaces are exposed to a wide variety of biological molecules, which can result in poisoning of catalytic activity or corrosion that can result in failure of the device. However, by utilizing the biocompatible membranes of the preferred embodiments in implantable devices, the active electrochemical surfaces of the sensor electrodes are preserved, and thus retain their activity for extended periods of time in vivo. By limiting access to the electrochemically reactive surface of the electrodes to a small number of molecular species, such as, for example, molecules having a molecular weight of about 34 Daltons (the molecular weight of peroxide) or less, only a small subset of the many molecular species present in biological fluids are permitted to contact the sensor. Use of such membranes enables the sustained function of devices for over one, two, three, or more years in vivo.

Biocompatible Membrane

The biocompatible membranes of preferred embodiments are constructed of two or more domains. The multi-domain membrane can be formed from one or more distinct layers and can comprise the same or different materials. The term “domain” is a broad term and is used in its ordinary sense, including, without limitation, a single homogeneous layer or region that incorporates the combined functions one or more domains, or a plurality of layers or regions that each provide one or more of the functions of each of the various domains.

FIG. 2 is an illustration of a biocompatible membrane in a preferred embodiment. The biocompatible membrane 18 can be used with a glucose sensor such, as is described above with reference to FIG. 1. In this embodiment, the biocompatible membrane 18 includes a cell disruptive domain 30 most distal of all membranes or layers from the electrochemically reactive surfaces, a cell impermeable domain 32 less distal from the electrochemically reactive surfaces than the cell disruptive domain, a resistance domain 34 less distal from the electrochemically reactive surfaces than the cell impermeable domain, an enzyme domain 36 less distal from the electrochemically reactive surfaces than the resistance domain, an interference domain 38 less distal from the electrochemically reactive surfaces than the enzyme domain, and an electrolyte domain 40 adjacent to the electrochemically reactive surfaces. However, it is understood that the biocompatible membrane can be modified for use in other devices, by including only two or more of the domains, or additional domains not recited above.

In some embodiments, all of the domains of the biocompatible membrane are formed from the silicone compositions described to above. In some embodiments, the biocompatible membrane is formed as a homogeneous membrane, namely, a membrane having substantially uniform characteristics from one side of the membrane to the other. However, a membrane can have heterogeneous structural domains, for example, domains resulting from the use of block copolymers (for example, polymers in which different blocks of identical monomer units alternate with each other), but can be defined as homogeneous overall in that each of the above-described domains functions by the preferential diffusion of some substance through the homogeneous membrane.

In some embodiments, one or more domains are formed from the silicone composition provided herein, while other domains are formed from other polymeric materials, for example, silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones and block copolymers thereof including, for example, di-block, tri-block, alternating, random and graft copolymers.

Cell Disruptive Domain

The cell disruptive domain 30 is positioned most distal to the electrochemically reactive surfaces and is designed to support tissue ingrowth, to disrupt contractile forces typically found in a foreign body capsule, to encourage vascularity within the membrane, and to disrupt the formation of a barrier cell layer. In one embodiment, the cell disruptive domain 30 has an open-celled configuration with interconnected cavities and solid portions, wherein the distribution of the solid portion and cavities of the cell disruptive domain includes a substantially co-continuous solid domain and includes more than one cavity in three dimensions substantially throughout the entirety of the first domain. Cells can enter into the cavities, however they cannot travel through or wholly exist within the solid portions. The cavities allow most substances to pass through, including, for example, cells, and molecules. U.S. patent application Ser. No. 09/916,386, filed Jul. 27, 2001, and entitled “MEMBRANE FOR USE WITH IMPLANTABLE DEVICES” and U.S. patent application Ser. No. 10/647,065, filed Aug. 22, 2003, and entitled, “POROUS MEMBRANES FOR USE WITH IMPLANTABLE DEVICES” describe membranes having a cell disruptive domain.

The cell disruptive domain 30 can be formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones or block copolymers thereof including, for example, di-block, tri-block, alternating, random and graft copolymers. In a preferred embodiment, the cell disruptive domain comprises a silicone composition of the preferred embodiments, for example, a silicone composition with a hydrophile such as Polyethylene Glycol (PEG) covalently incorporated or grafted therein. The PEG preferably includes from about 1 repeating unit to about 60 repeating units, more preferably from about 1, 2, 3, 4, 5, 6, 7, 8, 9, 10, 11, 12, 13, 14, or 15 repeating units to about 45, 46, 47, 48, 49, 50, 51, 52, 53, 54, 55, 56, 57, 58, or 50 repeating units, and most preferably from about 16, 17, 18, 19, 20, 21, 22, 23, 24, 25, 26, 27, 28, 29, or 30 repeating units to about 31, 32, 33, 34, 35, 36, 37, 38, 39, 40, 41, 42, 43, or 44 repeating units. Other hydrophiles that may be added to the silicone composition include, for example, other glycols such as propylene glycol, pyrrolidone, esters, amides, carbonates, and polypropylene glycol. In preferred embodiments, the PEG or other hydrophile comprises from about 0 wt. % to about 25, 30, 35, 40, 45, or 50 wt. % or more of the cell disruptive domain, more preferably from about 1 or 2 wt. % to about 10, 11, 12, 13, or 14 15, 16, 17, 18, 19, or 20 wt. %, and most preferably from about 3, 4, 5, or 6 wt. % to about 7, 8, or 9 wt. %. In preferred embodiments, the thickness of the cell disruptive domain is from about 10 or less, 20, 30, 40, 50, 60, 70, 80, or 90 microns to about 1500, 2000, 2500, or 3000 or more microns. In more preferred embodiments, the thickness of the cell disruptive domain is from about 100, 150, 200 or 250 microns to about 1000, 1100, 1200, 1300, or 1400 microns. In even more preferred embodiments, the thickness of the cell disruptive domain is from about 300, 350, 400, 450, 500, or 550 microns to about 500, 550, 600, 650, 700, 750, 800, 850, or 900 microns.

Cell Impermeable Domain

The cell impermeable domain 32 is positioned less distal to the electrochemically reactive surfaces than the cell disruptive domain, and is resistant to cellular attachment, is impermeable to cells, and is composed of a biostable material. Because the cell impermeable domain is resistant to cellular attachment (for example, attachment by inflammatory cells, such as macrophages, which are therefore kept a sufficient distance from other domains, for example, the enzyme domain), and because hypochlorite and other oxidizing species are short-lived chemical species in vivo, biodegradation does not occur. Additionally, the materials that are preferred to form this domain, for example, polycarbonate-based polyurethanes, silicones, and other such materials described herein, are resistant to the effects of these oxidative species and have thus been termed biodurable. See, e.g., U.S. patent application Ser. No. 09/916,386, filed Jul. 27, 2001, and entitled “MEMBRANE FOR USE WITH IMPLANTABLE DEVICES” and U.S. patent application Ser. No. 10/647,065, filed Aug. 22, 2003, and entitled, “POROUS MEMBRANES FOR USE WITH IMPLANTABLE DEVICES.”

The cell impermeable domain 32 may be formed from materials such as copolymers or blends of copolymers with hydrophilic polymers such as polyvinylpyrrolidone (PVP), polyhydroxyethyl methacrylate, polyvinylalcohol, polyacrylic acid, polyethers such as polyethylene glycol, and block copolymers thereof, including, for example, di-block, tri-block, alternating, random and graft copolymers (block copolymers are discussed in U.S. Pat. Nos. 4,803,243 and 4,686,044). In one preferred embodiment, the cell impermeable domain comprises a silicone composition of the preferred embodiments, for example a silicone composition with a hydrophile such as Polyethylene Glycol (PEG) covalently incorporated or grafted therein. The PEG preferably includes from about 1 repeating unit to about 60 repeating units, more preferably from about 1, 2, 3, 4, 5, 6, 7, 8, 9, 10, 11, 12, 13, 14, or 15 repeating units to about 45, 46, 47, 48, 49, 50, 51, 52, 53, 54, 55, 56, 57, 58, or 50 repeating units, and most preferably from about 16, 17, 18, 19, 20, 21, 22, 23, 24, 25, 26, 27, 28, 29, or 30 repeating units to about 31, 32, 33, 34, 35, 36, 37, 38, 39, 40, 41, 42, 43, or 44 repeating units. Other hydrophiles that may be added to the silicone composition include but are not limited to other glycols such as propylene glycol, pyrrolidone, esters, amides, carbonates, and polypropylene glycol. In preferred embodiments, the PEG or other hydrophile comprises from about 0 wt. % to about 25, 30, 35, 40, 45, or 50 wt. % or more of the cell impermeable domain, more preferably from about 1 or 2 wt. % to about 10, 11, 12, 13, 14, 15, 16, 17, 18, 19, or 20 wt. %, and most preferably from about 3, 4, 5, or 6 wt. % to about 7, 8, or 9] wt. %. In preferred embodiments, the thickness of the cell impermeable domain is from about 10 or 15 microns or less to about 125, 150, 175, or 200 microns or more. In more preferred embodiments, the thickness of the cell impermeable domain is from about 20, 25, 30, or 35 microns to about 65, 70, 75, 80, 85, 90, 95, or 100 microns. In even more preferred embodiments, the cell impermeable domain is from about 40 or 45 microns to about 50, 55, or 60 microns thick.

The cell disruptive domain 30 and cell impermeable domain 32 of the biocompatible membrane can be formed together as one unitary structure. Alternatively, the cell disruptive and cell impermeable domains 30, 32 of the biocompatible membrane can be formed as two layers mechanically or chemically bonded together.

Resistance Domain

The resistance domain 34 is situated more proximal to the electrochemically reactive surfaces relative to the cell disruptive domain. As described in further detail below, the resistance domain controls the flux of oxygen and glucose to the underlying enzyme domain. There exists a molar excess of glucose relative to the amount of oxygen in blood; that is, for every free oxygen molecule in extracellular fluid, there are typically more than 100 glucose molecules present (see Updike et al., Diabetes Care 5:207-21 (1982)). However, an immobilized enzyme-based sensor employing oxygen as cofactor is supplied with oxygen in non-rate-limiting excess in order to respond linearly to changes in glucose concentration, while not responding to changes in oxygen tension. More specifically, when a glucose-monitoring reaction is oxygen-limited, linearity is not achieved above minimal concentrations of glucose. Without a semipermeable membrane situated over the enzyme domain to control the flux of glucose and oxygen, a linear response to glucose levels can be obtained only up to about 40 mg/dL. However, in a clinical setting, a linear response to glucose levels is desirable up to at least about 500 mg/dL.

The resistance domain 34 includes a semipermeable membrane that controls the flux of oxygen and glucose to the underlying enzyme domain 36, preferably rendering oxygen in a non-rate-limiting excess. As a result, the upper limit of linearity of glucose measurement is extended to a much higher value than that which is achieved without the resistance domain. In one embodiment, the resistance domain 34 exhibits an oxygen-to-glucose permeability ratio of approximately 200:1. As a result, one-dimensional reactant diffusion is adequate to provide excess oxygen at all reasonable glucose and oxygen concentrations found in the subcutaneous matrix (See Rhodes et al., Anal. Chem., 66:1520-1529 (1994)). In some embodiments, a lower ratio of oxygen-to-glucose can be sufficient to provide excess oxygen by using an oxygen antenna domain (for example, a silicone material) to enhance the supply/transport of oxygen to the enzyme membrane. By enhancing the oxygen supply through the use of a silicone material, for example, a silicone composition of the preferred embodiments, glucose concentration may be less of a limiting factor. In other words, if more oxygen is supplied to the enzyme, then more glucose may also be supplied to the enzyme without creating an oxygen rate-limiting excess.

In a preferred embodiment, the resistance domain 34 comprises a silicone composition of the preferred embodiments, for example, a silicone composition with a hydrophile such as Polyethylene Glycol (PEG) covalently incorporated or grafted therein. Such resistance domains may be fabricated according to the method described above for forming films of the polymers of preferred embodiments. In one preferred embodiment, the resistance domain comprises a silicone composition of the preferred embodiments, for example, a silicone composition with a hydrophile such as Polyethylene Glycol (PEG) covalently incorporated or grafted therein. The PEG preferably includes from about 1 repeating unit to about 60 repeating units, more preferably from about 1, 2, 3, 4, 5, 6, 7, 8, 9, 10, 11, 12, 13, 14, or 15 repeating units to about 45, 46, 47, 48, 49, 50, 51, 52, 53, 54, 55, 56, 57, 58, or 50 repeating units, and most preferably from about 16, 17, 18, 19, 20, 21, 22, 23, 24, 25, 26, 27, 28, 29, or 30 repeating units to about 31, 32, 33, 34, 35, 36, 37, 38, 39, 40, 41, 42, 43, or 44 repeating units. Other hydrophiles that may be added to the silicone composition include but are not limited to other glycols such as propylene glycol, pyrrolidone, esters, amides, carbonates, and polypropylene glycol. In preferred embodiments, the PEG or other hydrophile comprises from about 0 wt. % to about 25, 30, 35, 40, 45, or 50 wt. % or more of the resistance domain, more preferably from about 1 or 2 wt. % to about 10, 11, 12, 13, 14, 15, 16, 17, 18, 19, or 20 wt. %, and most preferably from about 3, 4, 5, or 6 wt. % to about 7, 8, or 9 wt. %. In a particularly preferred embodiment, the resistance domain comprises 6 wt. % polyethylene glycol. By utilizing the silicone composition of the preferred embodiments, oxygen transport can be enhanced while glucose (or other analyte) can be sufficiently controlled.

In some embodiments, the resistance domain 34 can be formed as a unitary structure with the cell impermeable domain 32; that is, the inherent properties of the resistance domain 34 can provide the functionality described with reference to the cell impermeable domain 32 such that the cell impermeable domain 32 is incorporated as a part of resistance domain 24. In these embodiments, the combined resistance domain/cell impermeable domain can be bonded to or formed as a skin on the cell disruptive domain 30 during a molding process such as described above. In another embodiment, the resistance domain 34 is formed as a distinct layer and chemically or mechanically bonded to the cell disruptive domain 30 (when the resistance and cell impermeable domains are combined) or the cell impermeable domain 32 (when the resistance layer is distinct from the cell impermeable domain).

In preferred embodiments, the thickness of the resistance domain is from about 10 microns or less to about 200 microns or more. In more preferred embodiments, the thickness of the resistance domain is from about 15, 20, 25, 30, or 35 microns to about 65, 70, 75, 80, 85, 90, 95, or 100 microns. In more preferred embodiments, the thickness of the resistance domain is from about 40 or 45 microns to about 50, 55, or 60 microns.

Enzyme Domain

An immobilized enzyme domain 36 is situated less distal from the electrochemically reactive surfaces than the resistance domain 34. In one embodiment, the immobilized enzyme domain 36 comprises glucose oxidase. In other embodiments, the immobilized enzyme domain 36 can be impregnated with other oxidases, for example, galactose oxidase or uricase. For example, for an enzyme-based electrochemical glucose sensor to perform well, the sensor's response should neither be limited by enzyme activity nor cofactor concentration. Because enzymes, including glucose oxidase, are subject to deactivation as a function of ambient conditions, this behavior needs to be accounted for in constructing sensors for long-term use.

In certain preferred embodiments, the enzyme domain 36 comprises a silicone composition of the preferred embodiments wherein the silicone composition surrounds the enzyme. When the resistance domain 34 and enzyme domain 36 both comprise a silicone material (whether the silicone material composition is the same or different), the chemical bond between the enzyme domain 36 and resistance domain 34 is optimal, and the manufacturing made easy. Utilization of a silicone material, such as the silicone composition of the preferred embodiments, for the enzyme domain is also advantageous because silicone acts as an oxygen antenna domain and optimizes oxygen transport through the membrane to selected locations (for example, the enzyme membrane and/or counter electrode). The enzyme domain preferably comprises a silicone material of preferred embodiments and PEG. The PEG preferably includes from about 1 repeating unit to about 60 repeating units, more preferably from about 1, 2, 3, 4, 5, 6, 7, 8, 9, 10, 11, 12, 13, 14, or 15 repeating units to about 45, 46, 47, 48, 49, 50, 51, 52, 53, 54, 55, 56, 57, 58, or 50 repeating units, and most preferably from about 16, 17, 18, 19, 20, 21, 22, 23, 24, 25, 26, 27, 28, 29, or 30 repeating units to about 31, 32, 33, 34, 35, 36, 37, 38, 39, 40, 41, 42, 43, or 44 repeating units. Other hydrophiles that may be added to the silicone composition include but are not limited to other glycols such as propylene glycol, pyrrolidone, esters, amides, carbonates, and polypropylene glycol. In preferred embodiments, the PEG or other hydrophile comprises from about 0 wt. % to about 35, 40, 45, 50, 55, 60, 65, or 70 wt. % or more of the enzyme domain, more preferably from about 1, 2, or 3 wt. % to about 15, 16, 17, 18, 19, 20, 21, 22, 23, 24, 25, 26, 27, 28, 29, or 30 wt. %, and most preferably from about 4, 5, or 6 wt. % to about 7, 8, 9, 10, 11, 12, 13, or 14 wt. %. In a particularly preferred embodiment, the enzyme domain comprises 6 wt. % polyethylene glycol.

In an alternative embodiment, the enzyme domain 36 is constructed of aqueous dispersions of colloidal polyurethane polymers including the enzyme. In preferred embodiments, the thickness of the enzyme domain is from about 1 micron or less to about 40, 50, 60, 70, 80, 90, or 100 microns or more. In more preferred embodiments, the thickness of the enzyme domain is between about 1, 2, 3, 4, or 5 microns and 13, 14, 15, 20, 25, or 30 microns. In even more preferred embodiments, the thickness of the enzyme domain is from about 6, 7, or 8 microns to about 9, 10, 11, or 12 microns.

Interference Domain

The interference domain 38 is situated less distal to the electrochemically reactive surfaces than the immobilized enzyme domain. Interferants are molecules or other species that are electro-reduced or electro-oxidized at the electrochemically reactive surfaces, either directly or via an electron transfer agent, to produce a false signal (for example, urate, ascorbate, or acetaminophen). In one embodiment, the interference domain 38 prevents the penetration of one or more interferants into the electrolyte phase around the electrochemically reactive surfaces. Preferably, this type of interference domain is much less permeable to one or more of the interferants than to the analyte.

In one embodiment, the interference domain 38 can include ionic components incorporated into a polymeric matrix to reduce the permeability of the interference domain to ionic interferants having the same charge as the ionic components. In another embodiment, the interference domain 38 includes a catalyst (for example, peroxidase) for catalyzing a reaction that removes interferants. U.S. Pat. No. 6,413,396 and U.S. Pat. No. 6,565,509 disclose methods and materials for eliminating interfering species, however in the preferred embodiments any suitable method or material may be employed.

In another embodiment, the interference domain 38 includes a thin membrane that is designed to limit diffusion of species, e.g., those greater than 34 kD in molecular weight, for example. The interference domain permits analytes and other substances (for example, hydrogen peroxide) that are to be measured by the electrodes to pass through, while preventing passage of other substances, such as potentially interfering substances. In one embodiment, the interference domain 38 is constructed of polyurethane.

In a preferred embodiment, the interference domain 38 comprises a silicone composition. The interference domain preferably comprises a silicone material of preferred embodiments and PEG. The PEG preferably includes from about 1 repeating unit to about 60 repeating units, more preferably from about 1, 2, 3, 4, 5, 6, 7, 8, 9, 10, 11, 12, 13, 14, or 15 repeating units to about 45, 46, 47, 48, 49, 50, 51, 52, 53, 54, 55, 56, 57, 58, or 50 repeating units, and most preferably from about 16, 17, 18, 19, 20, 21, 22, 23, 24, 25, 26, 27, 28, 29, or 30 repeating units to about 31, 32, 33, 34, 35, 36, 37, 38, 39, 40, 41, 42, 43, or 44 repeating units. Other hydrophiles that may be added to the silicone composition include but are not limited to other glycols such as propylene glycol, pyrrolidone, esters, amides, carbonates, and polypropylene glycol. In preferred embodiments, the PEG or other hydrophile comprises from about 0 wt. % to about 11, 12, 13, 14, 15, 16, 17, 18, 19, or 20 wt. % or more of the enzyme domain, more preferably from about 1 wt. % to about 8, 9, or 10 wt. %, and most preferably from about 2 wt. % to about 3, 4, 5, 6, or 7 wt. %. In a particularly preferred embodiment, the interference domain comprises 6 wt. % polyethylene glycol. In preferred embodiments, the thickness of the interference domain is from about 0.1 microns or less to about 10 microns or more. In more preferred embodiments, the thickness of the interference domain is between about 0.2, 0.3, 0.4, or 0.5 microns and about 5, 6, 7, 8, or 9 microns. In more preferred embodiments, the thickness of the interference domain is from about 0.6, 0.7, 0.8, 0.9, or 1 micron to about 2, 3, or 4 microns.

Electrolyte Domain

An electrolyte domain 30 is situated more proximal to the electrochemically reactive surfaces than the interference domain 38. To ensure the electrochemical reaction, the electrolyte domain 30 includes a semipermeable coating that maintains hydrophilicity at the electrochemically reactive surfaces of the sensor interface. The electrolyte domain 40 enhances the stability of the interference domain 38 by protecting and supporting the material that makes up the interference domain. The electrolyte domain also 40 assists in stabilizing the operation of the device by overcoming electrode start-up problems and drifting problems caused by inadequate electrolyte. The buffered electrolyte solution contained in the electrolyte domain also protects against pH-mediated damage that may result from the formation of a large pH gradient between the substantially hydrophobic interference domain and the electrodes due to the electrochemical activity of the electrodes.

In one embodiment, the electrolyte domain 40 includes a flexible, water-swellable, substantially solid gel-like film having a “dry film” thickness of from about 2.5 microns to about 12.5 microns, more preferably from about 3, 3.5, 4, 4.5, 5, or 5.5 to about 6, 6.5, 7, 7.5, 8, 8.5, 9, 9.5, 10, 10.5, 11, 11.5, or 12 microns. “Dry film” thickness refers to the thickness of a cured film cast from a coating formulation onto the surface of the membrane by standard coating techniques.

In some embodiments, the electrolyte domain is formed of a curable mixture of a urethane polymer and a hydrophilic film-forming polymer. Particularly preferred coatings are formed of a polyurethane polymer having anionic carboxylate functional groups and non-ionic hydrophilic polyether segments, which is crosslinked in the presence of polyvinylpyrrolidone and cured at a moderate temperature of about 50° C.

In a preferred embodiment, the electrolyte domain 40 comprises a silicone composition of a preferred embodiment. The electrolyte domain preferably comprises a silicone material of preferred embodiments and PEG. The PEG preferably includes from about 1 repeating unit to about 60 repeating units, more preferably from about 1, 2, 3, 4, 5, 6, 7, 8, 9, 10, 11, 12, 13, 14, or 15 repeating units to about 45, 46, 47, 48, 49, 50, 51, 52, 53, 54, 55, 56, 57, 58, or 50 repeating units, and most preferably from about 16, 17, 18, 19, 20, 21, 22, 23, 24, 25, 26, 27, 28, 29, or 30 repeating units to about 31, 32, 33, 34, 35, 36, 37, 38, 39, 40, 41, 42, 43, or 44 repeating units. Other hydrophiles that can be added to the silicone composition include but are not limited to other glycols such as propylene glycol, pyrrolidone, esters, amides, carbonates, and polypropylene glycol. In preferred embodiments, the PEG or other hydrophile comprises from about 0 wt. % to about 25, 30, 35, 40, 45, 50, 55, 60, 65, or 70 wt. % or more of the electrolyte domain, more preferably from about 1, 2, or 3 wt. % to about 10, 11, 12, 13, 14, 15, 16, 17, 18, 19, 20 wt. %, and most preferably from about 4, 5, or 6 wt. % to about 7, 8, or 9 wt. %. In a particularly preferred embodiment, the electrolyte domain comprises 6 wt. % polyethylene glycol. In preferred embodiments, the thickness of the electrolyte domain is from about 1 micron or less to about 40, 50, 60, 70, 80, 90, or 100 microns or more. In more preferred embodiments, the thickness of the electrolyte domain is from about 2, 3, 4, or 5 microns to about 15, 20, 25, or 30 microns. In even more preferred embodiments, the thickness of the electrolyte domain is from about 6, 7, or 8 microns to about 9, 10, 11, or 12 microns.

Underlying the electrolyte domain is an electrolyte phase is a free-fluid phase including a solution containing at least one compound, typically a soluble chloride salt, which conducts electric current. In one embodiment wherein the biocompatible membrane is used with a glucose sensor such as is described herein, the electrolyte phase flows over the electrodes and is in contact with the electrolyte domain. The devices of the preferred embodiments contemplate the use of any suitable electrolyte solution, including standard, commercially available solutions. Generally, the electrolyte phase can have the same osmotic pressure or a lower osmotic pressure than the sample being analyzed. In preferred embodiments, the electrolyte phase comprises normal saline.

In various embodiments, any of these domains may be omitted, altered, substituted for, and/or incorporated together without departing from the spirit of the preferred embodiments. For example, because of the inherent properties of the silicone compositions of the preferred embodiments, a distinct cell impermeable domain may not exist. In such embodiments, other domains accomplish the function of the cell impermeable domain. As another example, the interference domain may be eliminated in certain embodiments wherein two-electrode differential measurements are employed to eliminate interference, for example, one electrode being sensitive to glucose and electrooxidizable interferants and the other only to interferants, such as is described in U.S. Pat. No. 6,514,718. In such embodiments, the interference layer may be omitted.

In general, the use of the silicone compositions of the preferred embodiments for some or all of the biocompatible membranes of an analyte sensor can result in numerous advantages. By forming one or more of the domains from the same or a similar silicone composition, the resulting membrane can be easily manufactured, securely bonded, and optimally designed. Another advantage of the silicone compositions of the preferred embodiments is that they can act as an oxygen reserve during times of minimal oxygen need and that they have the capacity to provide on demand a higher oxygen gradient to facilitate oxygen transport across the membrane, such as described in more detail below.

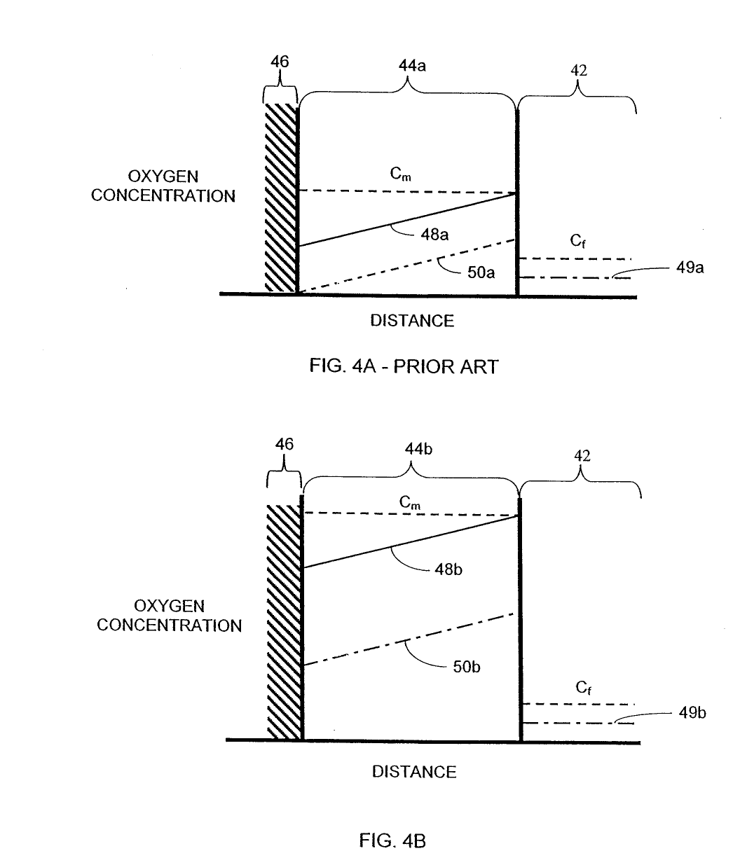

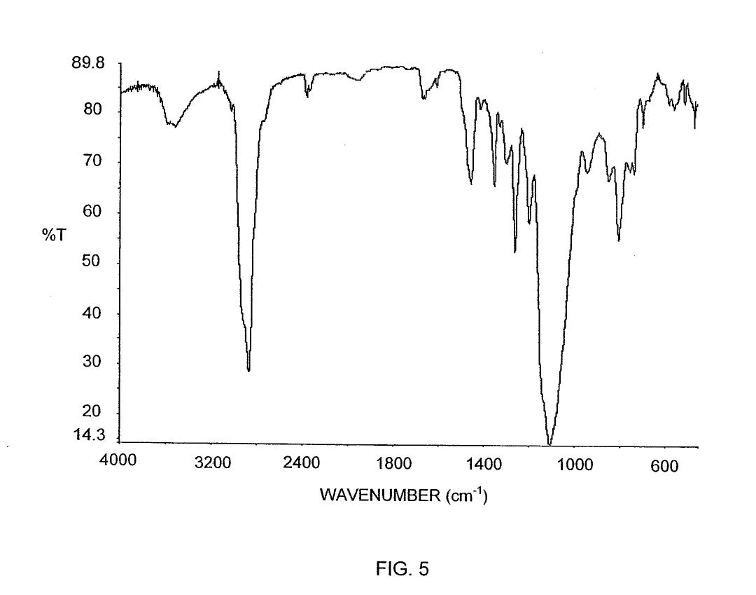

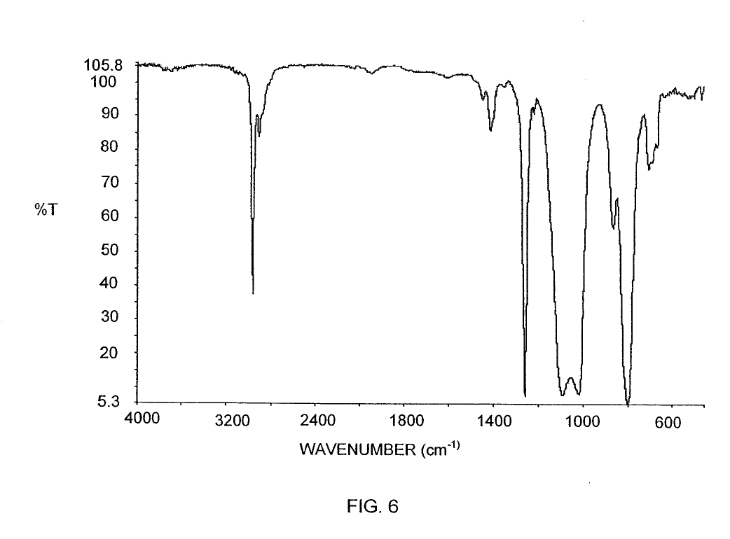

FIG. 4A is a schematic diagram of the oxygen concentration profiles of a conventional membrane. FIG. 4B is a schematic diagram of the oxygen concentration profiles of the biocompatible membrane of the preferred embodiments. In both diagrams, the x-axis represents distance and the y-axis represents oxygen concentration. These figures illustrate the difference between oxygen profiles of conventional (for example, prior art) biocompatible membranes versus oxygen profiles of the biocompatible membranes of the preferred embodiments. Namely, these figures illustrate the enhanced ability of the biocompatible membranes of the preferred embodiments to provide oxygen during transient ischemic periods.

Referring to FIG. 4A, a fluid source 42, such as interstitial fluid within the subcutaneous space, provides fluid to a biocompatible membrane 44a. The biocompatible membrane 44a is a conventional membrane, such as a polyurethane-based resistance membrane described in the Background Section. An oxygen-utilizing source 46, such as the enzyme domain described herein, utilizes oxygen from the fluid as a catalyst. In some alternative embodiments, the oxygen-utilizing source 46 comprises cells within a cell transplantation device, which utilize oxygen in the fluid for cellular processes. In some alternative embodiments, the oxygen-utilizing source 46 comprises an electro active surface that utilizes oxygen in an electrochemical reaction.

The upper dashed lines represent oxygen concentration in the fluid source (C_{f}) and oxygen concentration in the biocompatible membrane (Cₘ) at equilibrium (namely, without oxygen utilization) under normal conditions. However, when the biocompatible membrane 44a interfaces with an oxygen-utilizing source 46, oxygen concentration within the biocompatible membrane will be utilized. Accordingly, line 48a represents oxygen concentration under normal conditions decreasing at steady state as it passes through the biocompatible membrane 44a to the oxygen-utilizing source 46. While not wishing to be bound by theory, the oxygen concentration at the interface between the biocompatible membrane 44a and the oxygen-utilizing source 46 provides sufficient oxygen under normal conditions for oxygen-utilizing sources in vivo, such as enzymatic reactions, cellular processes, and electro active surfaces.

Unfortunately, “normal conditions” do not always occur in vivo, for example during transient ischemic periods, such as described in more detail above with reference to FIG. 2. During “ischemic conditions,” oxygen concentration is decreased below normal to a concentration as low as zero. Accordingly, line 49a represents oxygen concentration during an ischemic period, wherein the oxygen concentration of the fluid source (C_{f}) is approximately half of its normal concentration. It is noted that a linear relationship exists between the fluid source oxygen concentration (C_{f}) and the biocompatible membrane oxygen concentration (Cₘ) (see Hitchman, M. L. Measurement of Dissolved Oxygen. In Chemical Analysis; Elving, P., Winefordner, J., Eds.; John Wiley & Sons: New York, 1978; Vol. 49, pp. 63-70). Accordingly, line 50a represents the oxygen concentration within the biocompatible membrane during the ischemic period, which is approximately half of its normal concentration. Unfortunately, the resulting oxygen concentration at the interface of the membrane 44a and oxygen-utilizing source 46 is approximately zero. While not wishing to bound by theory, it is believed that the oxygen concentration at the interface between the conventional biocompatible membrane 44a and the oxygen-utilizing source 46 does not provide sufficient oxygen for oxygen-utilizing sources in vivo, such as enzymatic reactions, cellular processes, and electro active surfaces, during some ischemic conditions.

Referring to FIG. 4B, a fluid source 42, such as interstitial fluid within the subcutaneous space, provides fluid to a biocompatible membrane 44b. The biocompatible membrane 44b is a biocompatible membrane of the preferred embodiments, such as a resistance domain 34, a cell impermeable domain 32, and/or a cell disruptive domain 30 described herein, through which the fluid passes. An oxygen-utilizing source 46, such as the enzyme domain described herein, utilizes oxygen from the fluid as a catalyst. In some alternative embodiments, the oxygen-utilizing source 46 comprises cells within a cell transplantation device, which utilize oxygen in the fluid for cellular processes. In some alternative embodiments, the oxygen-utilizing source 46 comprises an electro active surface that utilizes oxygen in an electrochemical reaction.

The upper dashed lines represent oxygen concentration in the fluid source (C_{f}) and oxygen concentration in the biocompatible membrane (Cₘ) at equilibrium (namely, without oxygen utilization) under normal conditions. It is noted that the biocompatible membrane of the preferred embodiments 44b is illustrated with a significantly higher oxygen concentration than the conventional membrane 44a. This higher oxygen concentration at equilibrium is attributed to higher oxygen solubility inherent in the properties of the silicone composition of the preferred embodiments as compared to conventional membrane materials. Line 48b represents oxygen concentration under normal conditions decreasing at steady state as it passes through the biocompatible membrane 44b to the oxygen-utilizing source 46. While not wishing to be bound by theory, the oxygen concentration at the interface between the biocompatible membrane 44b and the oxygen-utilizing source 46 provides sufficient oxygen under normal conditions for oxygen-utilizing sources in vivo, such as enzymatic reactions, cellular processes, and electro active surfaces.

Such as described above, “normal conditions” do not always occur in vivo, for example during transient ischemic periods, wherein oxygen concentration is decreased below normal to a concentration as low as zero. Accordingly, line 49b represents oxygen concentration during ischemic conditions, wherein the oxygen concentration of the fluid source (C_{f}) is approximately half of its normal concentration. Because of the linear relationship between the fluid source oxygen concentration (C_{f}) and the biocompatible membrane oxygen concentration (Cₘ), the biocompatible membrane oxygen concentration, which is represented by a line 50b, is approximately half of its normal concentration. In contrast to the conventional membrane 50a illustrated in FIG. 4A, however, the high oxygen solubility of the biocompatible membrane of the preferred embodiments provides a reserve of oxygen within the membrane 44b, which can be utilized during ischemic periods to compensate for oxygen deficiency, illustrated by sufficient oxygen concentration 50b provided at the interface of the membrane 44b and oxygen-utilizing source 46. Therefore, the biocompatible membranes of the preferred embodiments provide an oxygen reserve that enables device function even during transient ischemic periods.

### EXPERIMENTS

The following examples illustrate the preferred embodiments. However, the particular materials, amounts thereof, and conditions recited in these examples should not be construed as limiting.

### Example 1

Size exclusion chromatography was performed on a system equipped with a Dynamax RI-1 detector, Waters 590 pump and two Shodex AT-80M/S columns in series. The system was calibrated using narrow molecular weight polystyrene standards whose M_{w}/Mₙ was less than 1.09. Samples were run in toluene at 4 ml/min and room temperature. FTIR spectra were collected on a PERKIN-ELMER 1600 Fourier-Transform Infrared spectrometer running in transmission mode. Samples were evaluated between KBr salt plates.

### Example 2

### Preparation of Cyclic Hydrophilic Monomer (Compound I)

To a 1 L three-necked round-bottomed flask were added tetramethylcyclotetrasiloxane (100 g, Gelest) and Pt-complex catalyst 2% in toluene (5 g, Aldrich). A thermometer, mechanical stirrer, heating mantle, pressure equalizing dropper funnel (500 ml), and a water cooled condenser were fitted to the flask. Heat was applied to the apparatus such that the flask temperature rose to and was held at about 70° to 80° C. Polyethyleneglycol allyl methyl ether (420 g, Clariant AM-250) was added dropwise to the flask over a period of fourteen hours. The reaction progress was monitored by observing the Si—H stretch (2163 cm⁻¹) in the FTIR spectrum. After no Si—H stretch was observed in the FTIR spectrum, the heating mantle was removed from the apparatus. The resulting yellow reaction mixture was allowed to cool to room temperature, and then was passed over a column (6″ tall, 1″ diameter) of activated aluminum oxide (Brockmann neutral, from Aldrich). In this way, 512 g of clear crude monomer (Compound I) was obtained. IR v: 3524, 2867, 1657, 1454, 1410, 1349, 1297, 1259, 1197, 1106, 943, 850, 803, 752, 735, 695, 556, 509, 465 cm⁻¹. The FTIR spectrum of Compound I is provided in FIG. 3.

### Example 3

### Preparation of Vinyl Terminated Silicone Copolymer (Polymer II)

To a 1 L three-necked round-bottomed flask were added octamethyl cyclotetrasiloxane (255.0 g, Gelest), hydrophilic monomer Compound I (30.0 g), toluene (150 ml, Aldrich) and vinyldimethylsilyl terminated polydimethylsiloxane (15.0 g, 200 cp, Andisil VS-200). The flask was fitted with a mechanical stirrer, a heating mantle, a thermometer, a Dean Stark trap, a water-cooled condenser, and a nitrogen source. Nitrogen was bubbled through the monomer solution for one hour. The flask was then heated to and held at 140° C. for 45 minutes. During this time, 20 ml of toluene was removed with the solvent trap. The reaction mixture was allowed to cool to 90° C. and a phosphazene base P₄-t-bu solution (15 μl, IM in hexanes, from Fluka) was added via syringe to the solution. The reaction mixture was stirred for 1 hour, after which the reaction temperature was reduced to room temperature. The resulting material was washed twice with methanol (300 ml, from Aldrich), then residual solvent was removed under reduced pressure. In this way, 246 g of Copolymer II was obtained, having M_{w}/Mₙ=490,000/195000. IR v: 3708, 2960, 2902, 1941, 1446, 1411, 1260, 1219, 1092, 1021, 864, 801, 702, 493, 462 cm⁻¹. The FTIR spectrum of Copolymer II is provided in FIG. 4.

The reaction scheme employed to prepare the vinyl-terminated silicone Copolymer II described above is as follows (Scheme 3):

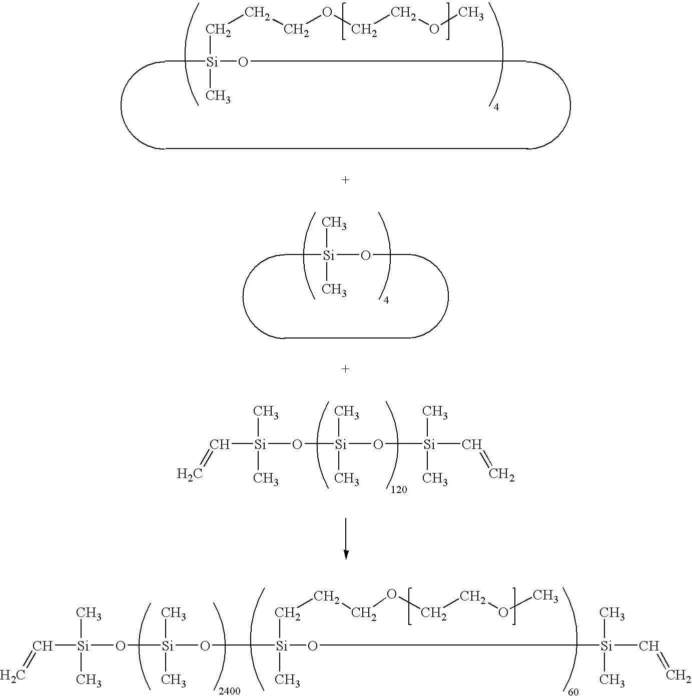

### Example 4

### Preparation of a Crosslinked Film

Into a 100 ml polyethylene mixing cup were placed vinyldimethylsilyl terminated polydimethylsiloxane (1.50 g, Andisil VS-20000), vinyl Q-resin (4.50 g, Andisil VQM 801), silicone Copolymer 11 (30.00 g), and treated fumed silica (12.00 g, Cabot CAB-O-SIL TS-530). This base rubber formulation was mixed at forty-five second intervals for a total of six minutes at 3500 rpm in a Hauschild Speed Mixer DAC 150 FV. The base rubber formulation was then allowed to cool to room temperature. Crosslinker (1.50 g, Andisil Crosslinker 200), chain extender (2.25 g, Andisil Modifier 705), and Pt catalyst (0.37 g, Andisil Catalyst 512 diluted to 33% in toluene) were compounded into the base rubber for forty-five seconds at 3500 rpm in the high-speed mixer. This material was diluted with toluene to 50% solids, and then coated onto TEFZEL® fluoropolymer film sold by DuPont (Wilmington, Del.) using a fixed gap (0.004″, Gardco 8-Path Applicator AP-15SS). Films were cured for one hour in a gravity oven set at 80° C.

### Example 5

### Glucose Testing

Membranes prepared under the conditions described in Example 4 were evaluated for their ability to allow glucose to permeate through the silicone composition. More specifically, a sensing membrane consisting of an enzyme layer, interference layer and electrode layer was affixed to six implantable analyte sensors, such as described in the section entitled, “Analyte Sensor”. In addition, three of the sensors (“Control”) were affixed most distally with a 50-micron thick silicone (NuSil MED-4840) membrane. The remaining three sensors (“Test”) were affixed most distally with a 50-micron thick silicone film prepared in Example 3. All sensors were allowed to equilibrate in phosphate buffered saline held at 37° C. The sensors were then exposed to 40, 200 and then 400 mg/dL glucose solutions for one hour each. The sensor signal was measured at each glucose concentration, and then plotted versus the glucose concentration. The best-fit line regressed through the data yields a slope that represents the glucose sensitivity of the sensors. Control sensor signals did not increase with exposure to glucose. However, the average glucose sensitivity for the test sensors was 14.2 pA per mg/dL of glucose with a standard deviation of 5.62 pA per mg/dL of glucose. Thus, the silicone composition test membranes allowed glucose to transport the membrane.

### Example 6

### Glucose Sensor Testing Under Varying Oxygen Concentrations

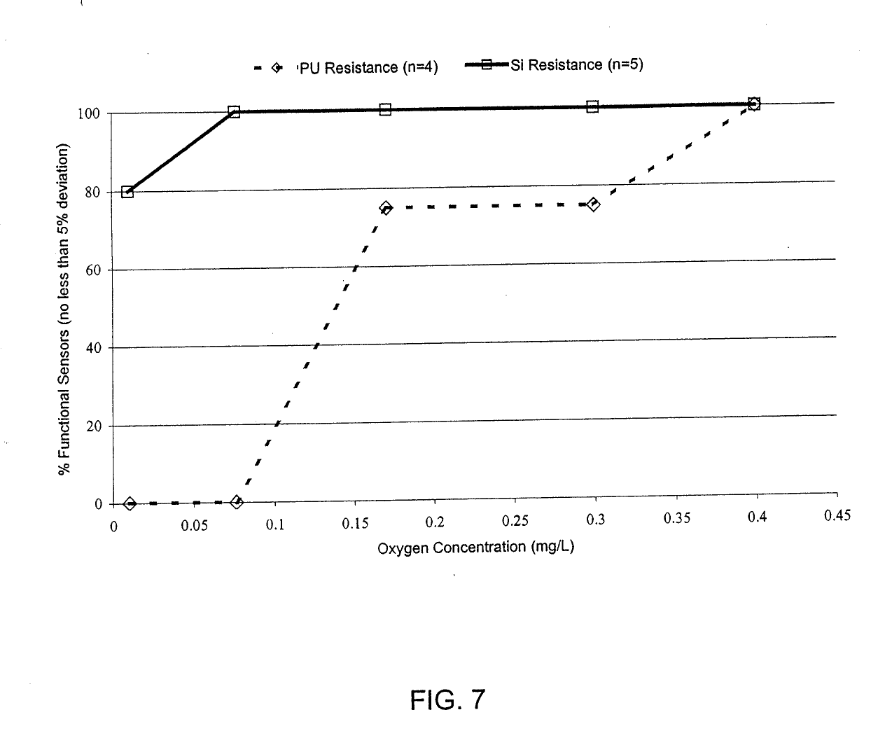

FIG. 7 is a graph that shows the results of an experiment comparing sensor function of sensors employing a conventional biocompatible membrane control versus sensors employing a biocompatible membrane of the preferred embodiments in simulated ischemic conditions. Both biocompatible membranes were comprised of a resistance domain, a polyurethane-based enzyme domain, and a polyurethane-based electrode domain as described herein. However, the conventional membranes comprised a conventional polyurethane-based resistance domain (“PU Resistance”) versus the biocompatible membranes of preferred embodiments, which comprised a resistance domain formed from a silicone composition of the preferred embodiments (“Si Resistance”) prepared under the conditions described in Example 4.

Four glucose sensors were affixed with conventional PU Resistance membranes for in vitro testing. Five glucose sensors were affixed with the preferred Si Resistance membrane for in vitro testing. All sensors were allowed to equilibrate in phosphate buffered saline held at 37° C. The sensors were then exposed to a glucose solution of 400 mg/dL and oxygen concentrations of 0.01, 0.076, 0.171, 0.3, and 0.4 mg/L were incrementally introduced into the solution using ratios of nitrogen gas and compressed air, returning to a oxygen concentration where sensors are fully functional (2 mg/dL) between each incremental test. The sensor signal was measured at each incremental oxygen concentration and the sensor considered functional if the signal deviation was no greater than 5% deviation from its measurement at normal oxygen concentration.

The percent of functional sensors in each group were plotted on the graph of FIG. 7 for each incremental oxygen concentration step. The vertical axis represents percent of functional sensors; the horizontal axis represents oxygen concentration in mg/dL. It is noted that at an oxygen concentration of 0.4 mg/L all sensors were functional. However, when oxygen concentration was decreased to 0.3 and 0.171 mg/L, some PU Resistance sensors failed to function within 5% deviation, while all Si Resistance sensors continued to function within 5% deviation. Finally, at the lowest oxygen concentration tests, 0.076 and 0.01 mg/L, none of the PU Resistance sensors functioned within 5% deviation, while the majority of the Si Resistance sensors continued to function within 5% deviation. While not wishing to be bound by theory, it is believed that the silicone composition of the preferred embodiments provides an oxygen reserve that supplements oxygen supply to a sensor or other device during transient ischemic conditions thereby decreasing oxygen limitation artifacts and increasing overall device function.

Methods and devices that are suitable for use in conjunction with aspects of the preferred embodiments are disclosed in copending U.S. application Ser. No. 10/648,849 filed Aug. 22, 2003 and entitled, “SYSTEMS AND METHODS FOR REPLACING SIGNAL ARTIFACTS IN A GLUCOSE SENSOR DATA STREAM”; U.S. application Ser. No. 10/646,333 filed Aug. 22, 2003 entitled, “OPTIMIZED SENSOR GEOMETRY FOR AN IMPLANTABLE GLUCOSE SENSOR”, now U.S. Pat. No. 7,134,999; U.S. application Ser. No. 10/647,065 filed Aug. 22, 2003 entitled, “POROUS MEMBRANES FOR USE WITH IMPLANTABLE DEVICES”; U.S. application Ser. No. 10/633,367 filed Aug. 1, 2003 entitled, “SYSTEM AND METHODS FOR PROCESSING ANALYTE SENSOR DATA”; U.S. application Ser. No. 09/916,386 filed Jul. 27, 2001 and entitled “MEMBRANE FOR USE WITH IMPLANTABLE DEVICES”, now U.S. Pat. No. 6,702,857; U.S. application Ser. No. 09/916,711 filed Jul. 27, 2001 and entitled “SENSOR HEAD FOR USE WITH IMPLANTABLE DEVICE”; U.S. application Ser. No. 09/447,227 filed Nov. 22, 1999 and entitled “DEVICE AND METHOD FOR DETERMINING ANALYTE LEVELS”; U.S. application Ser. No. 10/153,356 filed May 22, 2002 and entitled “TECHNIQUES TO IMPROVE POLYURETHANE MEMBRANES FOR IMPLANTABLE GLUCOSE SENSORS”; U.S. application Ser. No. 09/489,588 filed Jan. 21, 2000 and entitled “DEVICE AND METHOD FOR DETERMINING ANALYTE LEVELS”, now U.S. Pat. No. 6,741,877; U.S. application Ser. No. 09/636,369 filed Aug. 11, 2000 and entitled “SYSTEMS AND METHODS FOR REMOTE MONITORING AND MODULATION OF MEDICAL DEVICES”, now U.S. Pat. No. 6,558,321; and U.S. application Ser. No. 09/916,858 filed Jul. 27, 2001 and entitled “DEVICE AND METHOD FOR DETERMINING ANALYTE LEVELS,” now U.S. Pat. No. 6,862,465, as well as issued patents including U.S. Pat. No. 6,001,067 issued Dec. 14, 1999 and entitled “DEVICE AND METHOD FOR DETERMINING ANALYTE LEVELS”; U.S. Pat. No. 4,994,167 issued Feb. 19, 1991 and entitled “BIOLOGICAL FLUID MEASURING DEVICE”; and U.S. Pat. No. 4,757,022 filed Jul. 12, 1988 and entitled “BIOLOGICAL FLUID MEASURING DEVICE.”

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. All patents, applications, and other references cited herein are hereby incorporated by reference in their entirety.

1-33. (canceled)

34. An analyte sensor, comprising:
a working electrode; and
a biocompatible membrane disposed over the electrode, the biocompatible membrane comprising a silicone composition comprising a hydrophile incorporated therein, wherein the silicone composition is configured to resist diffusion of an analyte through the biocompatible membrane, and wherein the silicone composition comprises a silicone polymer whose backbone consists of alternating silicon and oxygen atoms and whose terminal groups are selected from the group consisting of alkyl, alkenyl, aryl and aralkyl moieties that are unsubstituted or substituted with one or more substituents selected from the group consisting of hydroxy, alkoxy, alkylsulfonyl, halogen, cyano, nitro, amino, and carboxyl, wherein the biocompatible membrane comprises a resistance domain, wherein the resistance domain controls a flux of oxygen and glucose through the membrane, and wherein the resistance domain comprises the silicone composition.

35. The analyte sensor of claim 34, wherein the silicone composition in the resistance domain comprises from about 1 wt. % to about 20 wt. % of the hydrophile.

36. The analyte sensor of claim 34, wherein the biocompatible membrane comprises an enzyme domain, wherein the enzyme domain comprises an immobilized enzyme.

37. The analyte sensor of claim 36, wherein the immobilized enzyme comprises glucose oxidase.

38. The analyte sensor of claim 36, wherein enzyme domain comprises a silicone composition comprising from about 1 wt. % to about 50 wt. % of the hydrophile.

39. The analyte sensor of claim 34, wherein the biocompatible membrane comprises an interference domain, wherein the interference domain substantially prevents the penetration of one or more interferents into an electrolyte phase adjacent to an electrochemically reactive surface.

40. The analyte sensor of claim 39, wherein the interference domain comprises an ionic component.

41. The analyte sensor of claim 39, wherein the interference domain comprises a silicone composition comprising from about 1 wt. % to about 10 wt. % of the hydrophile.

42. The analyte sensor of claim 34, wherein the biocompatible membrane comprises an electrolyte domain, wherein the electrolyte domain comprises a semipermeable coating that maintains hydrophilicity at an electrochemically reactive surface.

43. The analyte sensor of claim 42, wherein the electrolyte domain comprises a silicone composition comprising from about 1 wt. % to about 50 wt. % of the hydrophile.

44. The analyte sensor of claim 34, wherein the hydrophile is grafted therein.

45. The analyte sensor of claim 34, wherein the biocompatible membrane comprises two or more domains.

46. An analyte sensor, comprising:
a working electrode; and
a biocompatible membrane disposed over the electrode, the biocompatible membrane comprising a silicone composition comprising a hydrophile incorporated therein, wherein the silicone composition is configured to resist diffusion of an analyte through the biocompatible membrane, and wherein the silicone composition comprises a silicone polymer whose backbone consists of alternating silicon and oxygen atoms and whose terminal groups are selected from the group consisting of alkyl, alkenyl, aryl and aralkyl moieties that are unsubstituted or substituted with one or more substituents selected from the group consisting of hydroxy, alkoxy, alkylsulfonyl, halogen, cyano, nitro, amino, and carboxyl, wherein the silicone composition has an oxygen-to-analyte permeability ratio such that oxygen is provided to the immobilized enzyme in a non-rate-limiting excess for an enzyme-catalyzed reaction between oxygen and the analyte.

47. The analyte sensor of claim 46, wherein the oxygen-to-analyte permeability ratio is approximately 200:1.

48. The analyte sensor of claim 46, wherein the biocompatible membrane comprises a resistance domain, wherein the resistance domain comprises the silicone composition.

49. The analyte sensor of claim 48, wherein the silicone composition comprises a hydrophile covalently incorporated therein.

50. The analyte sensor of claim 48, wherein the silicone composition comprises from about 1 wt. % to about 19 wt. % of the hydrophile.

51. The analyte sensor of claim 48, wherein the silicone composition comprises from about 1 wt. % to about 10 wt. % of the hydrophile.

52. The analyte sensor of claim 48, wherein the silicone composition comprises from about 1 wt. % to about 8 wt. % of the hydrophile.

53. The analyte sensor of claim 46, wherein the hydrophile has a molecular weight from about 200 to about 1200 g/mol.

54. The analyte sensor of claim 46, wherein the analyte is glucose.

55. A continuous glucose sensor, comprising:
a working electrode configured to measure a signal associated with a concentration of glucose in a host; and
a biocompatible membrane disposed over the electrode, the biocompatible membrane comprising a silicone composition comprising a hydrophile incorporated therein, wherein the silicone composition is configured to resist diffusion of an analyte through the biocompatible membrane, and wherein the silicone composition comprises a silicone polymer whose backbone consists of alternating silicon and oxygen atoms and whose terminal groups are selected from the group consisting of alkyl, alkenyl, aryl and aralkyl moieties that are unsubstituted or substituted with one or more substituents selected from the group consisting of hydroxy, alkoxy, alkylsulfonyl, halogen, cyano, nitro, amino, and carboxyl, wherein the silicone composition is configured to resist diffusion of the analyte to an extent such that the sensor has a substantially linear response with respect to concentration of glucose up to glucose concentrations of at least about 500 mg/dL.

56. The analyte sensor of claim 55, wherein the silicone composition comprises from about 1 wt. % to about 20 wt. % of the hydrophile.

57. An analyte sensor, comprising:
a working electrode; and
a biocompatible membrane disposed over the electrode, the biocompatible membrane comprising a silicone composition comprising a hydrophile incorporated therein, wherein the silicone composition is configured to resist diffusion of an analyte through the biocompatible membrane, and wherein the silicone composition comprises a silicone polymer whose backbone consists of alternating silicon and oxygen atoms and whose terminal groups are selected from the group consisting of alkyl, alkenyl, aryl and aralkyl moieties that are unsubstituted or substituted with one or more substituents selected from the group consisting of hydroxy, alkoxy, alkylsulfonyl, halogen, cyano, nitro, amino, and carboxyl, wherein the biocompatible membrane comprises a cell disruptive domain, wherein the cell disruptive domain supports tissue ingrowth and interferes with barrier-cell layer formation, and wherein the cell disruptive domain comprises the silicone composition.

58. The analyte sensor of claim 57, wherein the silicone composition comprises from about 1 wt. % to about 20 wt. % of the hydrophile.

59. An analyte sensor, comprising:
a working electrode; and
a biocompatible membrane disposed over the electrode, the biocompatible membrane comprising a silicone composition comprising a hydrophile incorporated therein, wherein the silicone composition is configured to resist diffusion of an analyte through the biocompatible membrane, and wherein the silicone composition comprises a silicone polymer whose backbone consists of alternating silicon and oxygen atoms and whose terminal groups are selected from the group consisting of alkyl, alkenyl, aryl and aralkyl moieties that are unsubstituted or substituted with one or more substituents selected from the group consisting of hydroxy, alkoxy, alkylsulfonyl, halogen, cyano, nitro, amino, and carboxyl, wherein the biocompatible membrane comprises a cell impermeable domain, wherein the cell impermeable domain is resistant to cellular attachment and is impermeable to cells and cell processes, and wherein the cell impermeable domain comprises the silicone composition.

60. The analyte sensor of claim 59, wherein the silicone composition comprises from about 1 wt. % to about 20 wt. % of the hydrophile.
